SEQUENCE LISTING (2 sequences)

SEQ ID NO: 1 - protein, 9 aa, Artificial Sequence
  synthetic
RMFPNAPYL

SEQ ID NO: 2 - protein, 8 aa, Artificial Sequence
  synthetic
SIINFEKL

(19) United States
(12) Patent Application Publication
(10) Pub. No.: US 2020/0206333 A1
(43) Pub. Date: Jul. 2, 2020

(54) COMPOSITIONS FOR INDUCING AN IMMUNE RESPONSE

(71) Applicants: President and Fellows of Harvard College, Cambridge, MA (US); The General Hospital Corporation, Boston, MA (US)

(72) Inventors: Nisarg J. Shah, San Diego, CA (US); Ting-Yu Shih, Brookline, MA (US); Angelo S. Mao, Cambridge, MA (US); David J. Mooney, Sudbury, MA (US); David T. Scadden, Weston, MA (US)

(73) Assignees: President and Fellows of Harvard College, Cambridge, MA (US); The General Hospital Corporation, Boston, MA (US)

(21) Appl. No.: 16/708,218

(22) Filed: Dec. 9, 2019

Related U.S. Application Data
(63) Continuation of application No. PCT/US2018/036954, filed on Jun. 11, 2018.
(60) Provisional application No. 62/517,596, filed on Jun. 9, 2017.

(51) Int. Cl. A61K 39/00; A61K 39/39

(52) U.S. Cl. CPC A61K 39/001153; A61K 39/39; A61K 2039/55561; A61K 2039/804; A61K 31/704; A61K 2039/6087; A61K 2039/6093; A61K 2039/572; A61K 2039/545

(57) ABSTRACT

Acute myeloid leukemia (AML) is a clonal disorder of hematopoietic stem and progenitor cells. It is a devastating disease with a poor prognosis and an average 5-year survival rate of about 30%. Disclosed herein are composition and methods for treating leukemia with a biomaterial comprising a polymer scaffold, a dendritic cell activating factor, a dendritic cell recruitment factor, and at least one leukemia antigen. The biomaterial-based vaccine disclosed herein promotes a potent, durable and transferable immune response against acute myeloid leukemia to prevent cell engraftment and synergizes with chemotherapy to prevent relapse.

### CROSS-REFERENCE TO RELATED APPLICATIONS

This application is a continuation application of International Application No. PCT/US2018/036954, filed on Jun. 11, 2018, which claims the benefit of U.S. Provisional Application No. 62/517,596, filed on Jun. 9, 2017. The entire content of each of the foregoing applications are hereby incorporated by reference in their entirety.

### GOVERNMENT SUPPORT

This invention was made with government support under Grant Nos. U19HL129903, R01EB015498, and R01EB014703 awarded by the National Institutes of Health. The government has certain rights in the invention.

### REFERENCE TO SEQUENCE LISTING

This application incorporates by reference the Sequence Listing submitted in Computer Readable Form as file 117823-18802-SL.txt, created on Jan. 17, 2020 and containing 707 bytes.

### BACKGROUND OF THE INVENTION

Acute myeloid leukemia (AML) is a clonal disorder and malignancy of hematopoietic stem and progenitor cells (1, 2). It is a devastating disease with a poor prognosis and an average 5-year survival rate of about 30%. While there has been remarkable progress in the treatment of other chronic and acute leukemias, the standard-of-care treatment for AML, which consists of a cytotoxic chemotherapy of cytarabine and an anthracycline, has remained unchanged for over four decades (3). One striking observation with the current standard is that it generally reduces the AML burden and often induces a complete remission, but this therapeutic response is usually short-lived and rarely curative (4).

AML cells generally have a relatively low mutational load, are weak stimulators of host immune cells and often possess mechanisms that prevent induction of an effector T-cell response (5, 6). However, the recognition that leukemic blasts are susceptible to the graft-versus-leukemia (GvL) effect associated with allogeneic hematopoietic stem cell transplantation (HSCT) indicates the potential of harnessing the immune system to eradicate leukemia cells (7, 8). Genetic analysis has demonstrated that AML cells, like many other types of cancer cells, display tumor antigens that have the potential to trigger immune responses (9). Of the identified AML-associated antigens, Wilms Tumor protein-1 (WT-1) is a well-characterized intracellular zinc finger transcription factor with oncogenic potential (10). As a result of its overexpression in leukemias of multiple lineages, including in leukemic stem cell populations, and relative rarity in normal adult tissues, it is used as a prognostic biomarker (11). The Translational Research Working Group of the National Cancer Institute has ranked WT-1 as the highest priority cancer target for T-cell mediated immunotherapy (12).

To stimulate immune responses against AML, active immunization through vaccination has been tested in the clinic using single agent and combinations of WT-1, GM-CSF and dendritic cell-based vaccination approaches, which have been demonstrated to be safe (13). However, it has been observed that the immune response can be lost after repeated rounds of vaccination, likely due to the inefficient delivery of the vaccine components to the immune organs (14). In addition, the approach is not effective, as only a partial and transient effect has been observed in a small subset of patients. Thus, while the concept of therapeutically vaccinating patients against AML is attractive, there is a need to improve the robustness and durability of the immune response.
1. Shlush L I, Zandi S, Mitchell A, et al. Identification of pre-leukaemic haematopoietic stem cells in acute leukaemia. Nature. 2014; 506(7488):328-333.
2. Jan M, Snyder T M, Corces-Zimmerman M R, et al. Clonal evolution of preleukemic hematopoietic stem cells precedes human acute myeloid leukemia. Science translational medicine. 2012; 4(149):149ra118-149ra118.
3. Yates J W, Wallace H J, Jr., Ellison R R, Holland J F. Cytosine arabinoside (NSC-63878) and daunorubicin (NSC-83142) therapy in acute nonlymphocytic leukemia. Cancer chemotherapy reports. November-December 1973; 57(4):485-488.
4. Tallman M S, Gilliland D G, Rowe J M. Drug therapy for acute myeloid leukemia. Blood. Aug. 15, 2005; 106(4):1154-1163.
5. Barrett A, Le Blanc K. Immunotherapy prospects for acute myeloid leukaemia. Clinical & Experimental Immunology. 2010; 161(2):223-232.
6. Lawrence M S, Stojanov P, Polak P, et al. Mutational heterogeneity in cancer and the search for new cancer-associated genes. Nature. 2013; 499(7457):214-218.
7. Horowitz M M, Gale R P, Sondel P M, et al. Graft-versus-leukemia reactions after bone marrow transplantation. Blood. Feb. 1, 1990; 75(3):555-562.
8. H. W. Li, M. Sykes, Emerging concepts in haematopoietic cell transplantation. Nature Reviews Immunology 12, 403-416 (2012).
9. Anguille S, Van Tendeloo V F, Berneman Z N. Leukemia-associated antigens and their relevance to the immunotherapy of acute myeloid leukemia. Leukemia. October 2012; 26(10):2186-2196.
10. Rosenfeld C, Cheever M, Gaiger A. WT1 in acute leukemia, chronic myelogenous leukemia and myelodysplastic syndrome: therapeutic potential of WT1 targeted therapies. Leukemia. 2003; 17(7):1301-1312.
11. Dao T, Yan S, Veomett N, et al. Targeting the intracellular WT1 oncogene product with a therapeutic human antibody. Science translational medicine. Mar. 13, 2013; 5(176):176ra133.
12. Cheever M A, Allison J P, Ferris A S, et al. The prioritization of cancer antigens: a national cancer institute pilot project for the acceleration of translational research. Clinical cancer research: an official journal of the American Association for Cancer Research. Sep. 1, 2009; 15(17):5323-5337.
13. Grosso D A, Hess R C, Weiss M A. Immunotherapy in acute myeloid leukemia. Cancer. 2015; 121(16):2689-2704.
14. Rezvani K, Yong A S, Mielke S, et al. Leukemia-associated antigen-specific T-cell responses following combined PR1 and WT1 peptide vaccination in patients with myeloid malignancies. Blood. 2008; 111(1):236-242.

### SUMMARY OF THE INVENTION

Acute myeloid leukemia (AML) is a clonal disorder of hematopoietic stem and progenitor cells. It is a devastating disease with a poor prognosis and an average 5-year survival rate of about 30%. A shared hallmark in acute myeloid leukemia (AML) cells is the overexpression of leukemia-associated antigens, which represent promising targets for vaccination-based immunotherapy. To promote a robust and durable immune-response against AML, developed herein is a biomaterial-based injectable vaccine comprising encapsulated dendritic cell (DC) enhancement factor GM-CSF, DC activating factor CpG-ODN and a peptide antigen derived from Wilms tumor protein-1 (WT-1). WT-1 is an intracellular oncoprotein that is overexpressed in AML. The vaccines induced local infiltrates and activated DCs to evoke a potent anti-AML immune response.

Prophylactic vaccination with the disclosed biomaterial vaccine alone prevented the engraftment of AML cells. Combining chemotherapy and the biomaterial vaccine maximized efficacy to eradicate established disease. The combination treatment promoted antigen spreading, generated potent and durable long-term cellular responses, depleted leukemia-initiating cells, and immunized transplanted mice against AML. The results from an experimental mouse model of AML demonstrate the capacity of this biomaterial-based vaccination approach to provoke a potent immune response to eradicate AML and prevent relapse.

In some aspects, the invention is directed to a composition capable of inducing an endogenous immune response to leukemia (e.g., at least one leukemia antigen), comprising a polymer scaffold comprising open interconnected pores, a dendritic cell activating factor, a dendritic cell recruitment factor, and at least one leukemia antigen. In some embodiments, the at least one leukemia antigen is selected from the group consisting of Wilms' Tumor 1 protein (WT-1) or a fragment thereof, and leukemic bone marrow lysate. In some embodiments, the at least one leukemia antigen comprises WT-1 H-2Db peptide WT-1₁₂₆₋₁₃₄ (RMFPNAPYL (SEQ ID NO: 1)). In some aspects, the dendritic cell activating factor is CpG. In some embodiments, the dendritic cell activating factor is CpG 1826. In some embodiments, the dendritic cell recruitment factor is GM-CSF.

In some aspects, one or more of the dendritic cell activating factor, dendritic cell recruitment factor, and leukemia antigen are encapsulated by the polymer scaffold. In some embodiments, the polymer scaffold comprises polyethylene glycol (PEG) and alginate. In some embodiments, the polymer scaffold comprises a molar ratio of PEG to Alginate of about 1:4.

In some aspects of the invention, the composition is produced by cryo-polymerization of polymer components (e.g., MA-PEG and MA-Alginate) in the presence of one or more of the dendritic cell activating factor, dendritic cell recruitment factor, and leukemia antigen.

Another aspect of the invention is directed to administering the composition described above to a patient. In some embodiments, the patient has leukemia. In some embodiments, the leukemia is Acute Myeloid Leukemia (AML). In some embodiments, the patient is at risk of developing leukemia (e.g., AML). In some embodiments, the patient is in relapse. In some embodiments, the patient has undergone a procedure selected from a hematopoietic stem cell transplant, a T-cell therapy, and an adaptive immunity regimen.

In some embodiments of the method, the patient is also administered one or more anti-cancer agents before, after, or simultaneously with the composition. In some embodiments, the composition is administered immediately following an induction chemotherapy treatment. In some embodiments, the composition is administered within about 1 hour, 6 hours, 12 hours, 24 hours, 48 hours, 72 hours, 96 hours, 1 week, 2 weeks, or one month after an anti-cancer agent or treatment (e.g., induction chemotherapy). In some embodiments, the one or more cancer agents are administered about 1 day before administration of the composition. In some embodiments, the one or more anti-cancer agents are doxorubicin hydrochloride and cytarabine.

In some embodiments, the dendritic cell activating factor, the dendritic cell recruitment factor, and the at least one leukemia antigen release from the polymer scaffold over 30 days or less after administration to the patient. In some embodiments, at least one of the dendritic cell activating factor, the dendritic cell recruitment factor, and the at least one leukemia antigen burst release from the polymer scaffold after administration to the patient.

In some embodiments, the composition is administered by subcutaneous injection or implantation.

In some embodiments, administration of the composition induces cytotoxic T lymphocytes specific to leukemia in the patient. In some embodiments, administration of the composition induces an adaptive immune response specific to leukemia in the patient. In some embodiments, administration of the composition reduces or eliminates leukemia cells in the patient. In some embodiments, administration of the composition prevents or reduces the likelihood of a future occurrence of leukemia. In some embodiments, administration of the composition does not cause pancytopenia or autoimmunity in the subject.

Some aspects of the invention are directed to methods for preventing and/or reducing the incidence of leukemia in a subject, comprising transplanting bone marrow or hematopoietic stem cells from a donor to the subject, wherein the donor has been administered the composition described herein. In some embodiments, the subject has undergone myeloablation or myeloablative therapy prior to transplantation of bone marrow or hematopoietic stem cells from the donor. In some embodiments, the donor and the subject are different individuals. In some embodiments, the donor and subject are the same individual.

Some aspects of the invention are directed to a kit comprising the composition described herein.

### BRIEF DESCRIPTION OF THE DRAWINGS

These and other characteristics of the present invention will be more fully understood by reference to the following detailed description in conjunction with the attached drawings. The patent or application file contains at least one drawing executed in color. Copies of this patent or patent application publication with color drawings will be provided by the Office upon request and payment of the necessary fee.

FIGS. 1A-1K show that PEG-Alginate based cryogel vaccine sustains release of cytokines in vitro, preferentially accumulates and activates antigen-presenting cells in vivo. (FIG. 1A) Schematic for the covalently crosslinked cryogel vaccine loaded with cytokines and antigen, followed by subcutaneous injection. (FIG. 1B) In vitro release of GM-CSF (FIG. 1C) CpG and (FIG. 1D) antigen. (FIG. 1E) Measurement of the hydrogel injection site volume after subcutaneous injection over a period of 2 weeks (n=5). (*P<0.05, ** P<0.01, ***P<0.001, n.s., not significant (P>0.05), analysis of variance (ANOVA) with a Tukey post hoc test). (FIGS. 1F, G) Total number of recruited host cells (FIG. 1F) and CD11c⁺ dendritic cells (FIG. 1G) in a WT-1₁₂₆₋₁₃₄ cryogel vaccine (purple) or blank cryogel (black). (FIGS. 1H, I) Comparison of different cell types, including CD14⁺ monocytes, CD11c⁺ dendritic cells, B220⁺ B-cells and CD3⁺ T-cells contained within a WT-1₁₂₆₋₁₃₄ cryogel vaccine (FIG. 1H) or blank cryogel (FIG. 1I) up to 14 days post injection. (FIG. 1J) Numbers of CD11c⁺ CD86⁺ dendritic cells in dLNs after vaccination of the mice with the complete cryogel vaccine or a bolus subcutaneous injection of GM-CSF/CpG/antigen (n=5). (*P<0.05, ** P<0.01, ***P<0.001, analysis of variance (ANOVA) with a Tukey post hoc test). (FIG. 1K) Cell lysis as measured by the level of [3H]thymidine labeled DNA fragments from target cells in the presence and absence of effector cells at different CD8+ CTL: Target cell ratios. Symbols represent the mean lysis for the experiments shown. (*P<0.05, ** P<0.01, ***P<0.001, analysis of variance (ANOVA) with a Tukey post hoc test).

FIGS. 2A-2G show that prophylactic immunization with BM lysate and WT-1 peptide prevents AML engraftment. (FIG. 1A) Schedule of administration of the prophylactic vaccine, AML challenge and the monitoring of leukemia. (FIG. 1B) Representative FACS gating strategy for identifying WT-1 tetramer⁺ CD8⁺ T cells and IFN-γ⁺ CD8⁺ T-cells. (FIGS. 1C, D) The absolute number of WT-1 tetramer⁺ CD8⁺ T-cells (FIG. 1C) and IFN-γ⁺ CD8⁺ T-cells (FIG. 1D) in spleen, blood and bone marrow over the course of the study ((n=5 per group for each time point). (FIG. 1E) Representative bioluminescent images of AML progression in untreated and prophylactically immunized mice at Day 20. (FIG. 1F) Progression of AML in prophylactically treated study groups, measured as whole body radiance from luciferase expressing AML cells. Survival rate (FIG. 1G) after subcutaneous injection of various prophylactic vaccine formulations, AML challenge (Day 0) and Re-challenge (Day 100). Note: Both lysate and WT-1 vaccine groups showed no evidence of AML presence and the curve followed the X-axis (n=10 per group) (*P<0.05, ** P<0.01, ***P<0.001, n.s., not significant (P>0.05), analysis of variance (ANOVA) with a Tukey post hoc test).

FIGS. 3A-3F show that secondary transplants indicate the absence of AML initiating cells and the transference of immunity into transplant recipients. (FIG. 1A) GFP expression to monitor residual AML cells in bone marrow cells harvested from WT-1 prophylactically vaccinated mice and positive control of MLL-AF9 AML cells. (FIG. 1B) WT-1 tetramer⁺ CD8⁺ T cells in the harvested bone marrow cells from WT-1 prophylactically vaccinated animals and bone marrow from naïve mice. (FIG. 1C) Schedule of secondary transplant assay to determine leukemia initiating potential and transference of immunity. (FIG. 1D) Progression of AML measured as whole body radiance from luciferase expressing AML cells in transplanted mice (purple) or naïve mice injected with PBS as a negative control (blue). (FIG. 1E) IFN-γ⁺ CD8⁺ T cells in spleen and bone marrow of transplanted and naïve mice over the course of the study (n=5 per group for each time point) and survival rate (FIG. 1F) of naïve and transplanted mice after AML challenge (n=10 per group) (*P<0.05, ** P<0.01, ***P<0.001, n.s., not significant (P>0.05), analysis of variance (ANOVA) with a Tukey post hoc test).

FIGS. 4A-4J show that combination induction chemotherapy and cryogel vaccination with WT-1 eradicates established AML. (FIG. 1A) Timeline for AML establishment, administration of the treatments and monitoring of disease progression. Number of WT-1 tetramer⁺ CD8⁺ T-cells (FIG. 1B) and IFN-γ⁺ CD8⁺ T cells (FIG. 1C) in spleen, blood and bone marrow over the course of the study (n=5 per group for each time point). Imaging of AML in mice at day 21 (FIG. 1D), measured as whole body radiance from luciferase expressing AML cells (FIG. 1E) and survival rate in MLL/AF9)(FIG. 1F) and HoxA9-Meis1 (FIG. 1G) AML models (n=10 per group) (FIG. 1H). Expression of a subset of AML associated genes on Day 28 and Day 75 in AML cells harvested and pooled from the bone marrow, liver and spleen in relapsed mice. ovalbumin (OVA)—expressing AML cells (oAML) cells (5×10⁶) were injected into naïve mice i.v., and mice were treated and monitored as indicated (FIG. 1I). Staining with SIINFEKL-H-2K^{b} tetramers on peripheral blood mononuclear cells was performed on day 28. Shown are box plots (whiskers 5-95 percentile) (FIG. 1J) from one of two independent experiments (n=10 mice per group). (*P<0.05, ** P<0.01, ***P<0.001, n.s., not significant (P>0.05), analysis of variance (ANOVA) with a Tukey post hoc test).

FIGS. 5A-5F show secondary transplants indicate the absence of AML initiating cells and the transference of immunity into transplant recipients. (FIG. 1A) WT-1 tetramer⁺ CD8⁺ T cells in the harvested bone marrow cells from WT-1 prophylactically vaccinated animals and bone marrow from naïve mice. (FIG. 1B) Schedule of secondary transplant assay to determine leukemia initiating potential and transference of immunity. (FIG. 1C) Progression of AML measured as whole body radiance from luciferase expressing AML cells in transplanted mice (purple) or naïve mice injected with PBS as negative control (blue). (FIG. 1D) IFN-γ+ CD8+ T cells in spleen and bone marrow of transplanted and naïve mice over the course of the study (n=5 per group for each time point) and survival rate in transplants from MLL/AF9 (FIG. 1E) and HoxA9-Meis1 (FIG. 1F) animals (n=10 per group) (*P<0.05, ** P<0.01, ***P<0.001, n.s., not significant (P>0.05), analysis of variance (ANOVA) with a Tukey post hoc test).

FIGS. 6A-6D characterize immune reconstitution after hematopoietic stem cell transplant. (FIG. 6A) shows in vivo tracking of GFP-Luc expressing AML cells. As shown in (FIG. 6B), bioluminescence indicates efficacy of therapeutic and prophylactic vaccination strategies. (FIG. 6C) depicts loss of ovalbumin (OVA) expression in different hematopoietic compartments over time. (FIG. 6D) illustrates that both prophylactic and therapeutic vaccination strategies significantly increased survival (n=10 mice/group).

### DETAILED DESCRIPTION OF THE INVENTION

Some aspects of the invention are directed to a composition capable of inducing an endogenous immune response to leukemia (e.g., at least one leukemia antigen, at least two leukemia antigens, at least three leukemia antigens, or more), comprising a polymer scaffold, a dendritic cell activating factor, a dendritic cell recruitment factor, and at least one leukemia antigen. In some embodiments, the polymer scaffold (e.g., a three-dimensional polymer system) herein provides a delivery vehicle for the dendritic cell activating factor, the dendritic cell recruitment factor, and at least one leukemia antigen. In certain embodiments, the scaffold material is or comprises alginate (e.g., anionic alginate). In some embodiments, the scaffold material is in the form of a hydrogel. In some embodiments, the scaffold material is selected from the group consisting of polylactic acid, polyglycolic acid, PLGA polymers, alginates and alginate derivatives, polycaprolactone, calcium phosphate-based materials, gelatin, collagen, fibrin, hyaluronic acid, laminin rich gels, agarose, natural and synthetic polysaccharides, polyamino acids, polypeptides, polyesters, polyanhydrides, polyphosphazines, poly(vinyl alcohols), poly(alkylene oxides), poly(allylamines)(PAM), poly(acrylates), modified styrene polymers, pluronic polyols, polyoxamers, poly(uronic acids), poly(vinylpyrrolidone) and any combinations or copolymers thereof. Other exemplary scaffold materials, compositions and methods of their use and preparation are described in U.S. Patent Publication Nos. 2008/0044900, 2013/0331343 and 2015/0359928, which are incorporated by reference herein in their entirety.

In some embodiments, the scaffold material is a dendrimer. In some embodiments, the dendrimer comprises 1-99% of a first monomer and 1-99% of a second monomer. In some embodiments, the dendrimer comprises about 1-50% of a first monomer and 50-99% of a second monomer. In some embodiments, the dendrimer comprises about 20% of a first monomer and about 80% of a second monomer. In some embodiments, the first monomer is PEG (e.g., MA-PEG, PEG acrylate, 4 arm PEG acrylate) and the second monomer is alginate (e.g., MA-alginate). In some embodiments, the scaffold material (e.g., dendrimer) is a macroporous hydrogel consisting of, consisting essentially of, or comprising crosslinked polyethylene glycol (e.g., MA-PEG) and alginate (e.g., MA-Alginate). In some embodiments, the molar ratio of PEG to Alginate is about 1:1 to 1:10 or any ratio therebetween. In some embodiments, the molar ratio of PEG to Alginate is about 1:4.

The scaffold materials disclosed herein may be further modified, for example, to influence its mechanical properties. For example, to tune the mechanical properties of the scaffold material, polymers such as rigid polycaprolactone (PCL) and soft polyethylene glycol (PEG) can be used in combination with alginate.

In some embodiments, the scaffold material is in the form of a cryogel. Cryogels are a class of materials with a highly porous interconnected structure that are produced using a cryotropic gelation (or cryogelation) technique. Cryogelation is a technique in which the polymerization-crosslinking reactions are conducted in a quasi-frozen reaction solution. During freezing of the macromonomer (e.g., MA-alginate) solution, the macromonomers and initiator system (e.g., APS/TEMED) expelled from the ice concentrate within the channels between the ice crystals, so that the reactions only take place in these unfrozen liquid channels. After polymerization and, after melting of ice, a porous material is produced whose microstructure is a negative replica of the ice formed. Ice crystals act as porogens. Pore size is tuned by altering the temperature of the cryogelation process. For example, the cryogelation process is typically carried out by quickly freezing the solution at −20° C. Lowering the temperature to, e.g., −80° C., would result in more ice crystals and lead to smaller pores. In some embodiments, the cryogel is produced by cryo-polymerization of at least methacrylated (MA)-alginate and MA-PEG.

The cryogel may comprise at least 75% pores, e.g., 76%, 77%, 78%, 79%, 80%, 85%, 90%, 91%, 92%, 93%, 94%, 95%, 96%, 97%, 98%, or 99% or more pores. The pores are interconnected. Interconnectivity of the pores permits passage of water (and other compositions such as cells and compounds) in and out of the structure. In a fully hydrated state, the composition comprises at least 90% water (e.g., between 90-99%, at least 92%, 95%, 97%, 98%, or more) water. For example, at least 90% (e.g., at least 92%, 95%, 97%, 98%, or more) of the volume of the cryogel is made of liquid (e.g., water) contained in the pores. In a compressed or dehydrated hydrogel, up to 50%, 60%, 70% of that water is absent, e.g., the cryogel comprises less than 25% (e.g., less than 20%, 15%, 10%, 5%, or less) water.

The cryogels of the invention may comprise pores large enough for a cell to travel through. For example, the cryogel contains pores of 20-500 μm in diameter, e.g., about 20-300 μm, 30-150 μm, 50-500 μm, 50-450 μm, 100-400 μm, 200-500 μm in diameter. In some cases, the hydrated pore size is about 1-500 μm (e.g., bout 10-400 μm, 20-300 μm, or 50-250 μm). Methods for the preparation of polymer matrices having the desired pore sizes and pore alignments are described, e.g., in U.S. Pat. No. 6,511,650 and US Publication No. 2013/0202707, the entire contents of each of which is incorporated herein by reference.

In some embodiments, cryogels are further functionalized by addition of a functional group chosen from the group consisting of: amino, vinyl, aldehyde, thiol, silane, carboxyl, azide, alkyne. Alternatively, the cryogel is further functionalized by the addition of a further cross-linker agent (e.g. multiple arms polymers, salts, aldehydes, etc). The solvent can be aqueous, and in particular acidic or alkaline. The aqueous solvent can comprise a water-miscible solvent (e.g. methanol, ethanol, DMF, DMSO, acetone, dioxane, etc). In some embodiments, one or more functional groups are added to a constituent of the cryogel (e.g., alginate, PEG) prior to cryogelation. The cryo-crosslinking may take place in a mold and the injectable cryogels can be degradable. The pore size can be controlled by the selection of the main solvent used, the incorporation of a porogen, the freezing temperature and rate applied, the cross-linking conditions (e.g. polymer concentration), and also the type and molecule weight of the polymer used.

The scaffold materials may be used to control the in vivo presentation or release of a dendritic cell activating factor, a dendritic cell recruitment factor (e.g., granulocyte-macrophage colony-stimulating factor (GM-CSF)), and at least one antigen (e.g., leukemia antigen; leukemic bone marrow lysate), for example, upon administration or implantation of the scaffold material or composition. In some embodiments, the carboxylic acid group on the alginate backbone EDC/NHS chemistry is used to conjugate the dendritic cell activating factor, dendritic cell recruitment factor, and/or antigen to the scaffold material. Such presentation or release of one or more dendritic cell activating factors (e.g., unmethylated cytosine-guanosine oligodeoxynucleotide (CpG-ODN)), dendritic cell recruitment factors (e.g., GM-CSF), and/or antigens (e.g., leukemia antigen; WT-1 protein or fragment thereof, leukemic bone marrow lysate) may be accomplished by encapsulating or coupling (e.g., covalently binding or coupling) these molecules in or on the scaffold material (e.g., coupling the molecule to the alginate backbone). In some embodiments, the spatial and temporal presentation of such molecules is precisely controlled by fine-tuning the chemical reactions used to couple these molecules, as well as by selecting or altering the physical and chemical properties of the scaffold material. As a result, such scaffold materials are especially useful for controlling the in vivo delivery and/or presentation of one or more molecules that may be encapsulated therein or coupled thereto. In some embodiments, one or more dendritic cell activating factors (e.g., CpG-ODN), dendritic cell recruitment factors (e.g., GM-CSF), and/or antigens (e.g., leukemia antigen; WT-1 protein or fragment thereof, leukemic bone marrow lysate or a combination thereof) are encapsulated in a scaffold by cryo-polymerization of one or more polymers in the presence of the one or more dendritic cell activating factors, dendritic cell recruitment factors, and/or antigens. In some embodiments, one or more dendritic cell activating factors, one or more dendritic cell recruitment factors and one or more antigens are encapsulated in a scaffold by cryo-polymerization of one or more polymers in the presence of the one or more dendritic cell activating factors, one or more dendritic cell recruitment factors, and one or more antigens. In some embodiments, CpG-ODN, GM-CSF, and leukemia bone marrow lysate or a leukemia antigen (e.g., WT-1 protein or fragment thereof) are encapsulated in a scaffold by cryo-polymerization of one or more polymers in the presence of CpG-ODN, GM-CSF, and leukemia bone marrow lysate or a leukemia antigen (e.g., WT-1 or fragment thereof). In some embodiments, the one or more polymers are PEG and Alginate. In some embodiments, the antigen is WT-1 protein or fragment (e.g., antigenic fragment) thereof. In some embodiments, the WT-1 protein or fragment thereof is a WT-1 H-2Db peptide. In some embodiments, the WT-1 protein or fragment thereof is a WT-1 H-2Db peptide WT-1₁₂₆₋₁₃₄ (RMFPNAPYL (SEQ ID NO:1)),

In some embodiments, the leukemia antigen is AML1-ETO, DEK-CAN, PML-RARu, Flt3-ITD, NPM1, AurA, Bcl-2, BI-1, BMI1, BRAP, CML28, CML66, Cyclin B1, Cyclin E, CYP1B1, ETO/MTG8, G250/CAIX, HOXA9, hTERT, Mcl-1, Mesothelin, mHAg (eg, LRH-1), Myeloperoxidase, MPP11, MUC1, NuSAP1, OFA/iLRP, Proteinase 3, RGS5, RHAMM, SSX2IP, Survivin, WT-1, Cyclin A1, MAGE, PASD1, PRAME, or RAGE-1 or an antigenic fragment or antigenic derivative thereof. In some embodiments, the leukemia antigen is a WT-1 protein or antigenic fragment or antigenic derivative thereof. In some embodiments, the leukemia antigen is a proteinase-3 specific peptide (PR-1) or an antigenic fragment or antigenic derivative thereof. In some embodiments, the leukemia antigen is leukemic cell lysate. In some embodiments, the leukemic cell lysate is obtained from a candidate subject for performance of the methods of treatment disclosed herein.

WT1 gene (Wilms' tumor gene 1) has been identified as one of causative genes of Wilms' tumor, a childhood renal tumor (Cell 60: 509, 1990, Nature 343: 774, 1990). WT1 gene encodes the transcription factor WT-1, and WT-1 plays an important role in many processes such as proliferation, differentiation and apoptosis of cells, and development of tissues (Int. Rev. Cytol. 181: 151, 1998). The WT1 gene was originally defined as a tumor suppressor gene. However, subsequent studies revealed that WT-1 gene is expressed in leukemia and various solid cancers including lung cancer and breast cancer, indicating that WT1 gene rather exerts an oncogenic function promoting cancer growth. In addition, it was demonstrated in vitro that, when peripheral blood mononuclear cells positive for HLA-A*0201 or HLA-A*2402 are stimulated with WT-1-derived peptides, peptide-specific cytotoxic T-lymphocytes (CTLs) are induced and kill leukemia or solid tumor cells which endogenously express WT-1.

In some embodiments, the leukemia antigen is one described in Anguille, et al. “Leukemia-associated antigens and their relevance to the immunotherapy of acute myeloid leukemia,” Leukemia (2012) 26, 2186-2196. In some embodiments, the leukemia antigen is an antigen (e.g., neoantigen) present in leukemia of a candidate subject for administration of the compound. Any method of identifying a leukemia antigen may be used and is not limited. In some embodiments, the antigen is identified by sequencing the transcriptome of the candidate subject's leukemia cells.

In some aspects, the one or more dendritic cell activating factors is an antigen having a Pathogen-Associated Molecular Pattern (PAMP). In some embodiments, the PAMP antigen is a flagellin or a fragment or derivative thereof, a peptidoglycan or a fragment or derivative thereof, lipopolysaccharide (LPS) or a fragment or derivative thereof, double stranded RNA, or unmethylated DNA. In some embodiments, the one or more dendritic cell activating factors is an adjuvant. The term “adjuvant” encompasses substances that accelerate, prolong, or enhance the immune response to an antigen. In some embodiments an adjuvant serves as a lymphoid system activator that enhances the immune response in a relatively non-specific manner, e g., without having any specific antigenic effect itself. For example, in some embodiments an adjuvant stimulates one or more components of the innate immune system. In certain embodiments, an adjuvant enhances antigen-specific immune responses when used in combination with a specific antigen or antigens, e.g., as a component of a vaccine. Adjuvants include, but are not limited to, aluminum salts (alum) such as aluminum hydroxide or aluminum phosphate, complete Freund's adjuvant, incomplete Freund's adjuvant, surface active substances such as lysolecithin, pluronic polyols, Amphigen, Avridine, bacterial lipopolysaccharides, 3-O-deacylated monophosphoryl lipid A, synthetic lipid A analogs or aminoalkyl glucosamine phosphate compounds (AGP), or derivatives or analogs thereof (see, e.g., U.S. Pat. No. 6,113,918), L121/squalene, muramyl dipeptide, polyanions, peptides, saponins, oil or hydrocarbon and water emulsions, particles such as ISCOMS (immunostimulating complexes), etc. In some embodiments an adjuvant stimulates dendritic cell maturation. In some embodiments an adjuvant stimulates expression of one or more costimulator(s), such as B7 or a B7 family member, by antigen presenting cells (APCs), e.g., dendritic cells. In some embodiments an adjuvant comprises a CD40 agonist. In some embodiments, a CD40 agonist comprises an anti-CD40 antibody. In some embodiments, a CD40 agonist comprises a CD40 ligand, such as CD40L. In some embodiments an adjuvant comprises a ligand for a Toll-like receptor (TLR). In some embodiments, an agent is a ligand for one or more of TLRs 1-13, e.g., at least for TLR3, TLR4, and/or TLR9. In some embodiments, an adjuvant comprises a pathogen-derived molecular pattern (PAMP) or mimic thereof. In some embodiments, an adjuvant comprises an immunostimulatory nucleic acid, e.g., a double-stranded nucleic acid, e.g., double-stranded RNA or an analog thereof. For example, in some embodiments, an adjuvant comprises polyriboinosinic:polyribocytidylic acid (polyIC). In some embodiments an adjuvant comprises a nucleic acid comprising unmethylated nucleotides, e.g., a single-stranded CpG oligonucleotide. In some embodiments, an adjuvant comprises a cationic polymer, e.g., a poly(amino acid) such as poly-L-lysine, poly-L-arginine, or poly-L-ornithine. In some embodiments an adjuvant comprises a nucleic acid (e.g., dsRNA, polyIC) and a cationic polymer. For example, in some embodiments, an adjuvant comprises polyIC and poly-L-lysine. In some embodiments, an adjuvant comprises a complex comprising polyIC, poly-L-lysine, and carboxymethylcellulose (referred to as polyICLC). In some embodiments, an adjuvant comprises a CD40 agonist and a TLR ligand. For example, in some embodiments an adjuvant comprises (i) an anti-CD40 antibody and (ii) an immunostimulatory nucleic acid and/or a cationic polymer. In some embodiments, an adjuvant comprises an anti-CD40 antibody, an immunostimulatory nucleic acid, and a cationic polymer. In some embodiments, an adjuvant comprises (i) an anti-CD40 antibody and (ii) poly(IC) or poly(ICLC). In certain embodiments, an adjuvant is pharmaceutically acceptable for administration to a human subject. In certain embodiments an adjuvant is pharmaceutically acceptable for administration to a non-human subject, e.g., for veterinary purposes.

In some embodiments, the dendritic cell activating factor is CpG (i.e., CpG-ODN). The CpG may be of Class A or Class B. In some embodiments, the CpG is CpG 2006, CpG 1968, or CpG 1826. In some embodiments, the dendritic cell activating factor is CpG 1826.

In some embodiments, the dendritic cell recruitment factor is GM-CSF or a fragment or derivative thereof. In some embodiments, the dendritic cell recruitment factor is SDF-1 or a fragment or derivative thereof.

In some embodiments, the composition has a volume of about 1-500 μL (e.g., 10-250 μL, 20-100 μL, 40-60 μL, or about 50 μL). In some embodiments, the composition contains about 0.01 to 100 μg, about 0.1 to 10 μg, or about 1 μg dendritic cell recruitment factor. In some embodiments, the composition contains about 0.1 μg to 10 mg, about 1 μg to 1 mg, about 10 μg to 500 μg, or about 100 μg dendritic cell activating factor. In some embodiments, the composition contains about 0.1 μg to 10 mg, about 1 μg to 1 mg, about 10 μg to 500 μg, or about 100 μg antigen (e.g., WT-1 protein or fragment thereof). In some embodiments, the composition contains about 1-10 parts by weight of dendritic cell recruitment factor to about 10-1000 parts by weight of dendritic cell activating factor and about 10-1000 parts by weight of antigen. In some embodiments, the composition contains about 1 part by weight of dendritic cell recruitment factor to about 100 parts by weight of dendritic cell activating factor and about 100 parts by weight of antigen. In some embodiments, the composition contains about 1 part by weight of GM-CSF to about 100 parts by weight of CpG and about 100 parts by weight of WT-1 or fragment thereof (i.e., a ratio of 1:100:100 GM-CSF:CpG:WT-1 or fragment thereof).

The compositions of the invention exhibit sustained release of one or more of the dendritic cell recruitment factors, dendritic cell activating factors and antigens over a period of days, weeks or months upon administration to a patient. In some embodiments, the period of sustained release is about 1-5, 1-10, 1-20, 1-30, 1-50, 1-100 days, or more. In some embodiments, about 5%, 10%, 15%, 20%, 25%, 30%, 35%, 40%, 45%, 50%, 55%, 60%, 65%, 70%, 75%, 80%, 85%, 90%, 95%, or 100% of one or more of the dendritic cell recruitment factors, dendritic cell activating factors and antigens exhibit sustained release.

In some embodiments, at least a portion of the one or more of the dendritic cell recruitment factors, dendritic cell activating factors and antigens burst release from the composition upon administration to a patient. In some embodiments, about 1% to 50% of the one or more of the dendritic cell recruitment factors, dendritic cell activating factors, and antigens burst release from the composition upon administration to a patient. In some embodiments, about 1% to 25% of one or more of the dendritic cell recruitment factors, dendritic cell activating factors and antigens burst release from the composition upon administration to a patient. In some embodiments, about 1% to 10% of the one or more of the dendritic cell recruitment factors, dendritic cell activating factors and antigens burst release from the composition upon administration to a patient. In some embodiments, about 8% of the dendritic recruitment factor is burst released upon administration to a patient. In some embodiments, about 3%-3.5% of the antigen is burst released upon administration to a patient. In some embodiments, the burst release occurs within 1 hour, 6 hours, 12 hours, 1 day, 2 days, 3 days, or more. In some embodiments, extended release of one or more of the dendritic cell recruitment factors, dendritic cell activating factors and antigens occurs after burst release.

In some embodiments, the composition is an immunogenic composition (also referred to as a “vaccine composition”) that generates or stimulates an immune response ex vivo or in vivo.

### Methods of Treating Leukemia

Some aspects of the invention are directed towards methods of treating leukemia in a patient in need thereof, comprising administering the compositions described herein.

As used herein, a “patient” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “subject” and “patient” are used interchangeably herein. In some embodiments, the subject suffers from acute myeloid leukemia (AML). In some embodiments, the subject suffers from AML and is a poor candidate for Hematopoietic Stem Cell Transplant (HSCT). In some embodiments, the patient has received HSCT. In some embodiments, the patient has received induction chemotherapy. In some embodiments, the patient received or is receiving a T-cell therapy or an adaptive immunity technique.

As used herein, the term “treating” and “treatment” refers to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment.

In some embodiments, the leukemia is selected from the group consisting of acute myeloid leukemia (AML), myelodysplastic syndrome (MDS), acute lymphoblastic leukemia (ALL) and chronic lymphocytic leukemia (CLL). In some embodiments, the leukemia is acute myeloid leukemia. As used herein, “acute myeloid leukemia” encompasses all forms of acute myeloid leukemia and related neoplasms according to the World Health Organization (WHO) classification of myeloid neoplasms and acute leukemia, including all of the following subgroups in their relapsed or refractory state: Acute myeloid leukemia with recurrent genetic abnormalities, such as AML with t(8;21)(q22;q22); RUNX1-RUNX1T1, AML with inv(16)(p13.1q22) or t(16;16)(p13.1;q22); CBFB-MYH11, AML with t(9;11)(p22;q23); MLLT3-MLL, AML with t(6;9)(p23;q34); DEK-NUP214, AML with inv(3)(q21 q26.2) or t(3;3)(q21;q26.2); RPN1-EVI1, AML (megakaryoblastic) with t(1;22)(p13;q13); RBM15-MKL1, AML with mutated NPM1, AML with mutated CEBPA; AML with myelodysplasia-related changes; therapy-related myeloid neoplasms; AML, not otherwise specified, such as AML with minimal differentiation, AML without maturation, AML with maturation, acute myelomonocytic leukemia, acute monoblastic/monocytic leukemia, acute erythroid leukemia (e.g., pure erythroid leukemia, erythroleukemia, erythroid/myeloid), acute megakaryoblastic leukemia, acute basophilic leukemia, acute panmyelosis with myelofibrosis; myeloid sarcoma; myeloid proliferations related to Down syndrome, such as transient abnormal myelopoiesis or myeloid leukemia associated with Down syndrome; and blastic plasmacytoid dendritic cell neoplasm.

As used herein, the method of administering is not limited. In some embodiments, the compositions described herein are administered, e.g., implanted, e.g., orally, systemically, sub- or trans-cutaneously, as an arterial stent, surgically, or via injection. In some examples, the compositions described herein are administered by routes such as injection (e.g., subcutaneous, intravenous, intracutaneous, percutaneous, or intramuscular) or implantation.

In some embodiments, the compositions described herein are injected. In some embodiments, the composition is injectable through a 16-gauge, an 18-gauge, a 20-gauge, a 22-gauge, a 24-gauge, a 26-gauge, a 28-gauge, a 30-gauge, a 32-gauge, or a 34-gauge needle. In some embodiments, upon compression or dehydration, the composition maintains structural integrity and shape memory properties, i.e., after compression or dehydration, the composition regains its shape after it is rehydrated or the shear forces of compression are removed/relieved. In some embodiments, the composition also maintains structural integrity in that it is flexible (i.e., not brittle) and does not break under sheer pressure. In some embodiments, the composition is injected subcutaneously.

In some embodiments, the composition is administered once every day to once every 10 years (e.g., once every day, once every week, once every two weeks, once every month, once every two months, once every 3 months, once every 4 months, once every 5 months, once every 6 months, once every year, once every 2 years, once every 3 years, once every 4 years, once every 5 years, once every 6 years, once every 7 years, once every 8 years, or once every 10 years). In other examples, the composition is administered once to 5 times (e.g., one time, twice, 3 times, 4 times, 5 times, or more as clinically necessary) in the subject's lifetime.

In some embodiments, the methods of the invention further comprise administering one or more anti-cancer agents (e.g., chemotherapeutic agents) to the patient.

Chemotherapeutic agents useful in methods, compositions, and/or kits disclosed herein include, but are not limited to, alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, bendamustine, chlorozotocin, fotemustine, lomustine, nimustine, ranimustine; antibiotics such as aclacinomysins, actinomycin, authramycin, azaserine, bleomycins, dactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel and docetaxel; chlorambucil; gemcitabine; 6-thioguanine; mercaptopurine; platinum analogs such as cisplatin and carboplatin; vinblastine; platinum; etoposide; ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine; retinoic acid; esperamicins; capecitabine; and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide, and irinotecan, as well as pharmaceutically acceptable salts, acids, or derivatives of any of these. In some embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain e embodiments, the antimitotic agent comprises a vinca alkaloid, such as vincristine, binblastine, vinorelbine, or vindesine, or pharmaceutically acceptable salts, acids, or derivatives thereof.

In some embodiments, the one or more anti-cancer agents are cytarabine and an anthracycline. In some embodiments, the one or more anti-cancer agents are doxorubicin hydrochloride and cytarabine.

In some embodiments, the one or more anti-cancer agents are administered prior to, simultaneously with, or after administration of the compositions of the invention. In some embodiments, the one or more anti-cancer agents are administered about 1, 2, 3, 4, 5, 6, 7, 8, 9, 10, 15, 20, 25, 30, 60, 90, 120 days prior to, or after, the administration of the composition.

In some embodiments of the invention, the composition is administered to a subject reduce or eliminate the likelihood of developing leukemia (e.g., AML). In some embodiments, the subject has an increased risk of developing leukemia (e.g., AML). Several inherited genetic disorders and immunodeficiency states are associated with an increased risk of AML. These include disorders with defects in DNA stability, leading to random chromosomal breakage, such as Bloom's syndrome, Fanconi's anemia, Li-Fraumeni kindreds, ataxia-telangiectasia, and X-linked agammaglobulinemia. In some embodiments, the subject has increased risk of developing leukemia (e.g., AML) due to age (e.g., over about 60, 65, 70, 75, 80 years or more). In some embodiments, the subject has already been treated for leukemia (e.g., AML) and is in relapse. In some embodiments, the subject is treated by the methods disclosed herein immediately (e.g., within about 1 day, 2 days, 3 days, 4 days, 1 week, 2 weeks, 3 weeks, 1 month) after induction chemotherapy.

In some embodiments, administration of the composition reduces the risk of developing leukemia by about 2-fold, 3-fold, 4-fold, 5-fold, or more. In some embodiments, the administration of the composition reduces the risk of developing leukemia (e.g., AML) by about 10%, 20%, 30%, 40%, 50%, 60%, 70%, 80%, 90%, 95%, 99%, or more.

In some embodiments, administration of the composition reduces the risk of developing leukemia (e.g., AML) for about 3 months, 6 months, 9 months, 1 year, 2 years, 3 years, 4 years, 5 years, 7 years, 10 years, 15 years or more.

In some embodiments, administration of the composition to a patient having leukemia or at risk of developing leukemia increases the number of CD11c+ cells. In some embodiments, administration of the composition increases the number of CD11c+ cells by about 2-fold, 3-fold, 4-fold, 5-fold, or more. In some embodiments, the administration of the composition increases the number of CD11c+ cells by about 10%, 20%, 30%, 40%, 50%, 60%, 70%, 80%, 90%, 95%, 100%, 200%, 300%, 400%, or more.

In some embodiments, administration of the composition to a patient having leukemia or at risk of developing leukemia does not increase the risk of developing pancytopenia and/or autoimmunity.

In some embodiments, administration of the composition to a patient having leukemia or at risk of developing leukemia induces immunostimulation against leukemia and/or long term immunity to leukemia (e.g., AML).

Some aspects of the invention are directed to a method for preventing and/or reducing the incidence of leukemia in a subject, comprising transplanting bone marrow or hematopoietic stem cells from a donor to the subject, wherein the donor has been administered the composition described herein. In some embodiments, the hematopoietic stem cells have been obtained from a donor subjected to a mobilization regimen to increase hematopoietic stem cells in the peripheral blood.

### Compositions and Kits

Described herein are kits for practicing methods disclosed herein and for making compositions disclosed herein. In some aspects, a kit includes at least a composition comprising a polymer scaffold comprising open interconnected pores, a dendritic cell activating factor, a dendritic cell recruitment factor, and at least one leukemia antigen.

Each of the polymer scaffold, dendritic cell activating factor, dendritic cell recruitment factor, and leukemia antigen may be any described herein. In some embodiments, the kit comprises a polymer scaffold as described herein encapsulating CpG-ODN, GM-CSF and WT-1 H-2Db peptide WT-1₁₂₆₋₁₃₄. In some embodiments, the kit comprises components (e.g., monomers) for producing a polymer scaffold as described herein, a dendritic cell activating factor, a dendritic cell recruitment factor, and at least one leukemia antigen. In some embodiments, the kit comprises one or more reagents for forming a polymer scaffold from components (e.g., monomers) as described herein.

In any embodiments, one or more components of the kit may be supplied in a watertight or gas tight container which in some embodiments is substantially free of other components of the kit. The kit components can be supplied in more than one container. In some embodiments, one or more kit components can be provided in liquid, dried or lyophilized form. In some embodiments, one or more components of the kit are substantially pure and/or sterile. When a component described herein is provided in a liquid solution, the liquid solution preferably is an aqueous solution, with a sterile aqueous solution being preferred. When a component described herein is provided as a dried form, reconstitution generally is by the addition of a suitable solvent. The solvent, e.g., sterile water or buffer, can optionally be provided in the kit.

In some embodiments, the kit further optionally comprises information material. The informational material can be descriptive, instructional, marketing or other material that relates to the methods described herein and/or the use of a compound(s) described herein for the methods described herein.

The informational material of the kits is not limited in its instruction or informative material. In one embodiment, the informational material can include information about production of the compound, molecular weight of the compound, concentration, date of expiration, batch or production site information, and so forth. In one embodiment, the informational material relates to methods for administering the compound. Additionally, the informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In one embodiment, the informational material can include instructions to administer a composition as described herein in a suitable manner to perform the methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer a composition described herein to a suitable subject, e.g., a human, e.g., a human having or at risk for a disorder described herein or to a cell in vitro.

In some embodiments, the kit includes a plurality (e.g., a pack) of individual containers, each containing one or more unit dosage forms (e.g., a dosage form described herein) of a composition described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe or any such delivery device.

Specific examples of the inventions disclosed herein are set forth below in the Examples.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, cell, or non-human transgenic animal, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Example 1

Our work and that of others has demonstrated that certain biomaterials are useful in enhancing the effectiveness of vaccines and other immunotherapies (15-20). In this study, we sought to determine if a durable anti-AML immune response could be elicited using a biomaterial-based vaccine to both prevent AML engraftment and to synergize with chemotherapy. We previously reported the design and assembly of macroporous biomaterials that activate host immune cells in vivo, and their utility in vaccination against solid tumors (21-24). Based on these results, we hypothesized that similar success could be achieved for AML with a biomaterial-based vaccine containing AML-associated antigens. To this end, a macroporous hydrogel was constructed using a combination of polyethylene glycol and alginate as the scaffold material, encapsulated AML associated antigens, the TLR-9 agonist cytosine-guanosine oligodeoxynucleotide (CpG) as the adjuvant, and granulocyte-macrophage colony-stimulating factor (GM-CSF) to recruit and proliferate dendritic cells (25, 26). The scaffold induced the trafficking of innate immune cells, which included host antigen presenting cells, presented AML-associated antigens and ultimately led to robust T-cell responses. In two syngeneic AML mouse models derived from fusion oncoproteins—MLL/AF9 and HoxA9/Meis1, the cryogel vaccine alone prevented the engraftment of AML cells. Furthermore, the vaccine in combination with the standard-of-care chemotherapy regimen eradicated established AML and elicited long-lived and transferable protective T-cell memory responses in immunocompetent mice.

Results

Synthesis and Assembly of a Biomaterial-Based AML Vaccine

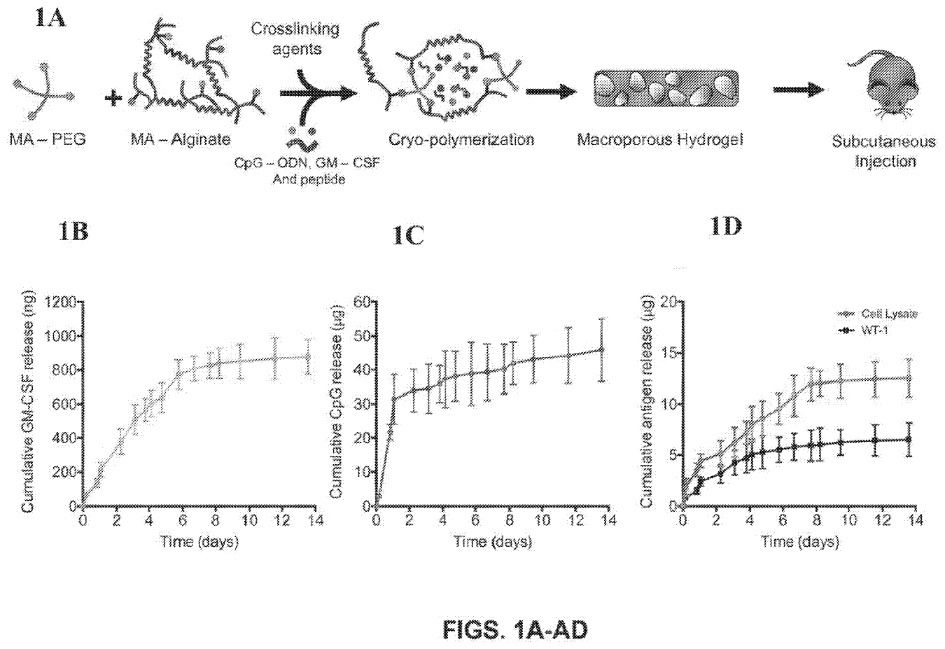

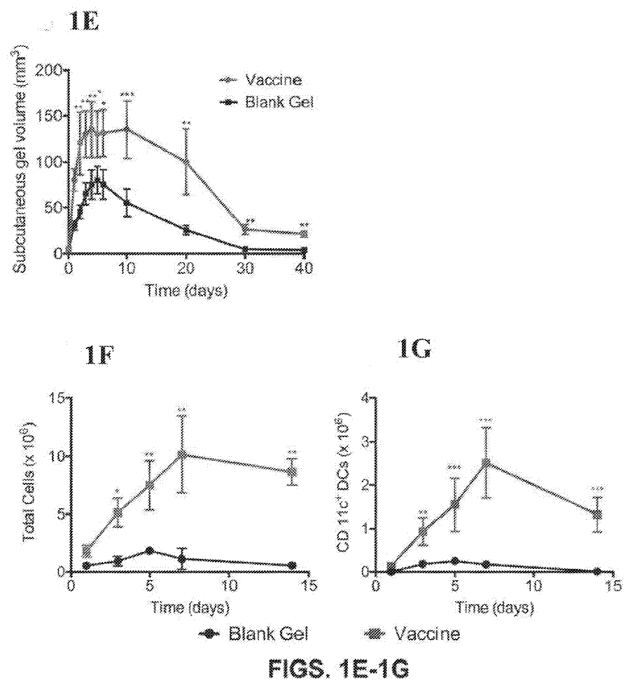

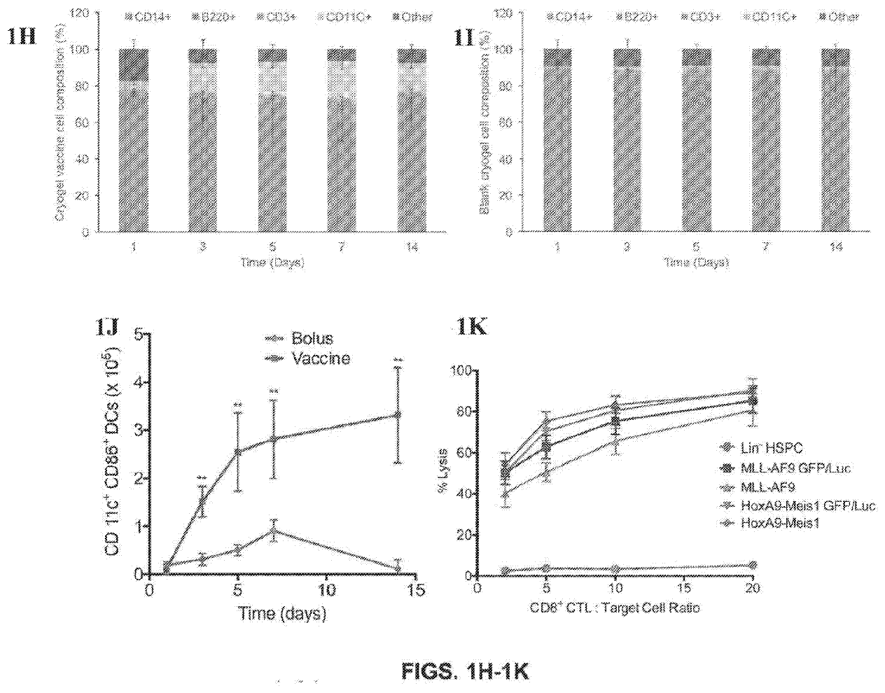

A macroporous hydrogel consisting of crosslinked methacrylated polyethylene glycol (MA-PEG) and methacrylated alginate (MA-Alginate) (Molar ratio: 1:4) was constructed using a previously reported cryo-polymerization technique. Prior to the initiation of cryo-polymerization, 1 μg of the cytokine granulocyte-macrophage colony-stimulating factor (GM-CSF) and 100 μg of unmethylated cytosine-guanosine oligodeoxynucleotide (CpG-ODN 1826) were added to the mixture of MA-PEG and MA-Alginate. AML-associated antigens in the form of either 100 μg of freeze-thaw cell lysates from the bone marrow of terminally-ill mice with AML or 100 μg of WT-1 H-2Db peptide WT-1₁₂₆₋₁₃₄ (RMFPNAPYL (SEQ ID NO:1)) was added to the mixture. The cryo-polymerization process was intended to encapsulate the biomolecules in the resulting macroporous hydrogel, referred to as the vaccine cryogel (FIG. 1A). GM-CSF (encapsulation efficiency 87%), CpG-ODN (encapsulation efficiency 48%) and antigen release (cell lysate encapsulation efficiency 77%; WT-1₁₂₆₋₁₃₄ encapsulation efficiency 75%), was subsequently assayed by sandwich enzyme-linked immunosorbent assay (ELISA), Oligreen assay and micro bicinchoninic acid (micro-BCA) assay respectively. After a burst release of about 8% of the loaded amount, GM-CSF eluted in a sustained manner. 85% of the GM-CSF was released over the first 5 days in vitro (FIG. 1B). 50% of the CpG-ODN eluted from the hydrogel within the first 2 days, followed by sustained release at a slower rate (FIG. 1C). It has been previously demonstrated that both GM-CSF and CpG-ODN, that are encapsulated and released in a similar manner, retain their bioactivity (>80%) in vitro (21). After a burst release of 3.3% of the WT-1₁₂₆₋₁₃₄ or 8% of the loaded cell lysates, over the first two days, the antigens released in a sustained manner (FIG. 1D). Approximately 9 μg of the cell lysates and 4 μg of the WT-1₁₂₆₋₁₃₄ released over a period of 10 days after the burst release.

Spatiotemporal Characterization of Innate Immune Cell Trafficking

The macroporous cryogel was next analyzed for its ability to induce the trafficking of host innate immune cells. Vaccine or blank cryogels, which did not contain the encapsulated GM-CSF, CpG-ODN and AML-associated antigen, were subcutaneously injected in 6-8 week old C57BL/6 mice. To grossly quantify infiltration in the blank and vaccine cryogel after the injection, each subcutaneous nodule was measured over a period of 6 weeks (FIG. 1E). In the cryogel vaccine, nodule size rapidly increased in size over the first 5 days, growing to approximately 25 times the initial volume, followed by size reduction to 3-4 times the initial volume by day 40. In contrast, the blank scaffolds increased to approximately 15 times the initial volume and reduced to the original volume over the same period. The cryogels and cells in the draining lymph nodes (dLN) were harvested from mice and analyzed over a period of 2 weeks to quantify the dynamics of cell trafficking. The number of cells present in the vaccine cryogel was 3- and 9-fold higher at day 1 and 7, respectively, compared to the blank cryogel (FIG. 1F). The number of CD11c+ cells was significantly higher at all time points (FIG. 1G, ANOVA with a Tukey post hoc test; n=5 per group per time point).

Detailed analysis of the cell composition indicated a peak between days 5 and 7 (FIG. 1H, FIG. 1I) in the number of CD11c+ cells that were present in the vaccine cryogel. At day 5, the vaccine cryogel contained CD11c+ cells (18%), B220+ B cells (9%) and CD14+ monocytes (62%). In contrast, most of the cells in the blank cryogel were CD14+ cells (>80%) at all timepoints. Bolus vaccination, which consisted of an intraperitoneal (i.p.) injection of a combined GM-CSF, CpG and WT-1₁₂₆₋₁₃₄ (same doses as included in cryogel vaccine) in phosphate buffered saline (PBS), resulted in significantly lower numbers of CD11c+ CD86+ activated dendritic cells in the dLN at all of the time points that were analyzed when compared to the vaccine cryogel (FIG. 1J, ANOVA with a Tukey post hoc test; n=5 per group per time point).

Cytotoxic T Lymphocyte Recognition of WT-1 in AML Cells

To examine whether AML cells could be lysed by WT-1 specific CTL, splenocytes were isolated from prophylactically vaccinated mice, 10 days after vaccination. MLL-AF9 and HoxA9-Meis1 cells were each susceptible to lysis by the WT-1 specific CTL in in vitro, whereas lineage depleted hematopoietic stem and progenitor cells were no susceptible to a cytotoxic response (FIG. 1K). The cytotoxic response was similar in the transgenic and GFP-luciferase expressing AML cell variants.

Prophylactic Vaccination Prevents MLL-AF9 AML Cell Engraftment

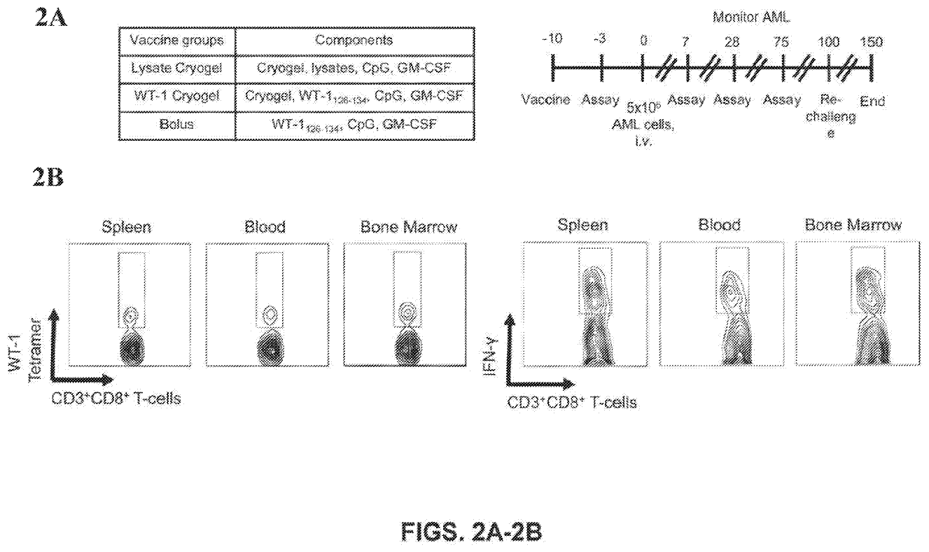

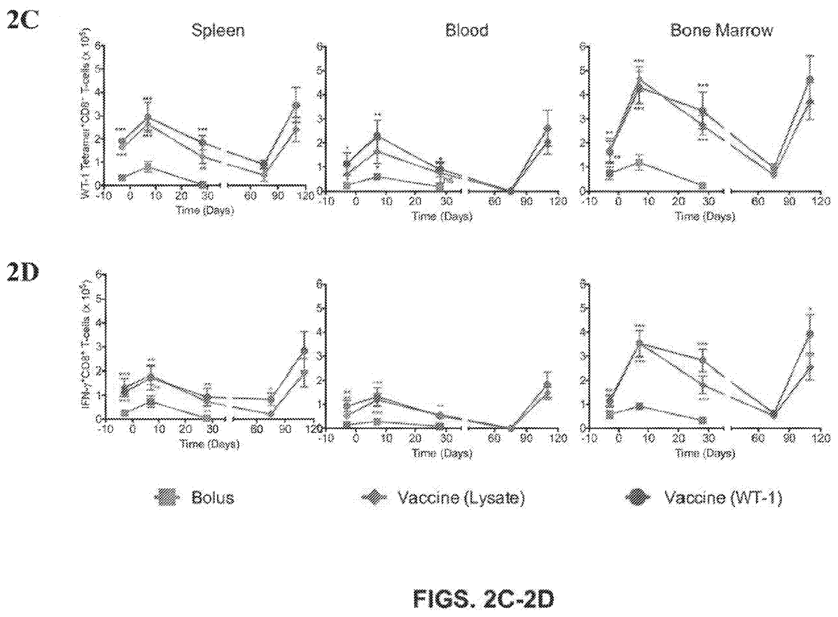

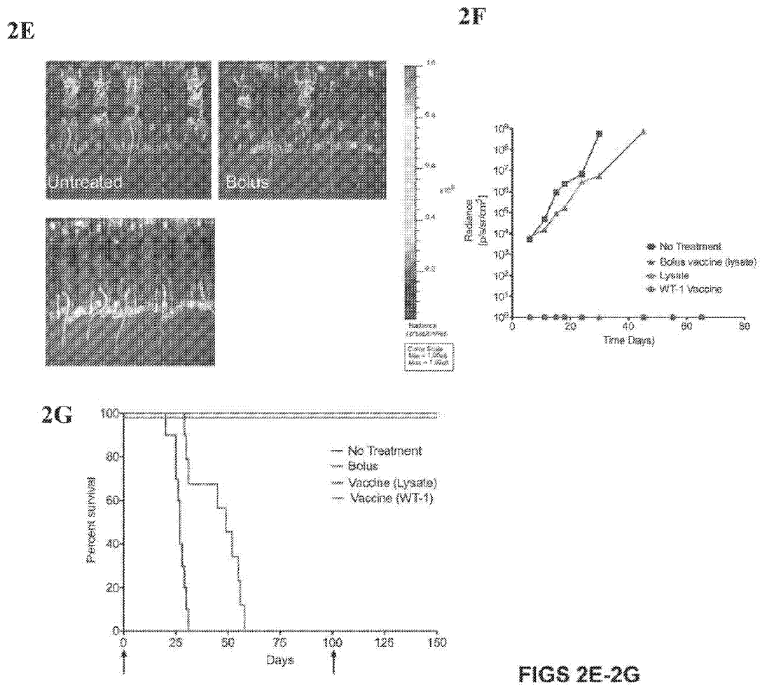

Next, the induction of an antigen-specific adaptive immune response by the cryogel vaccine was studied. To confer prophylactic protection against the engraftment of AML cells, the cryogel vaccine was administered to enhance the CD8⁺ cytotoxic T-lymphocyte (CTL) immune response against WT-1. C57Bl/6 mice were immunized using (i) vaccine cryogel with cell lysates as the antigen, (ii) vaccine cryogel with WT-1₁₂₆₋₁₃₄ as the antigen or (iii) bolus vaccine with WT-1₁₂₆₋₁₃₄ as the antigen (FIG. 2A). The strength of the CD8⁺ T-cell response was measured by analyzing (i) the frequency of antigen-specific RMFPNAPYL (SEQ ID NO: 1) tetramer⁺ CD8⁺ T cells and (ii) IFN-γ⁺CD8⁺ T-cells after in vitro peptide re-stimulation for a functional readout of CTLs from the blood, spleen and bone marrow, which constitute the hematopoietic compartments in which AML cells are commonly observed. Significantly higher numbers of WT-1 tetramer⁺ CD8⁺ T-cells (FIG. 2B, C) and IFN-γ⁺CD8⁺ T cells (FIG. 2B, D) were found in cryogel vaccinated mice, compared with mice receiving the bolus vaccine (ANOVA with a Tukey post hoc test; n=5 per group per time point).

We investigated the effect of cryogel vaccination in providing prophylactic protection against the MLL/AF9 AML. Mice were immunized with cryogel vaccines or controls and subsequently challenged with an intravenously injection of 5 million cells (viability >95%). One hundred days after the primary AML challenge, vaccinated mice were re-challenged with 5 million MLL-AF9 AML cells. An increase in antigen-specific CD8⁺ T-cells following re-challenge mirrored a corresponding increase in IFN-γ secreting CD8⁺ T-cells in the blood, spleen and bone marrow (FIG. 2C, D). The cryogel vaccine with either the cell lysates or WT-1₁₂₆₋₁₃₄ as the antigen conferred full protection against the primary AML challenge and the subsequent re-challenge in all vaccinated mice. The GFP-luciferase reporter in the AML cell line was used to measure the AML burden in live animals over the duration of the study (FIG. 2D, E). AML cells were initially detected in the long bones of untreated and bolus vaccine treated mice at the same time point (FIG. 2E). Thereafter, the progression of AML accelerated in untreated mice, which predictably succumbed to the AML between days 23 and 29 post-challenge (FIG. 2F). The bolus vaccine slowed the progression of AML and significantly increased the survival (log-rank test; n=10 per group), and mice succumbed between days 49 and 59 post-challenge. There were no detectable levels of AML cells observed in cryogel vaccinated mice.

Prophylactic Cryogel Vaccination Depletes Leukemia Initiating Cells

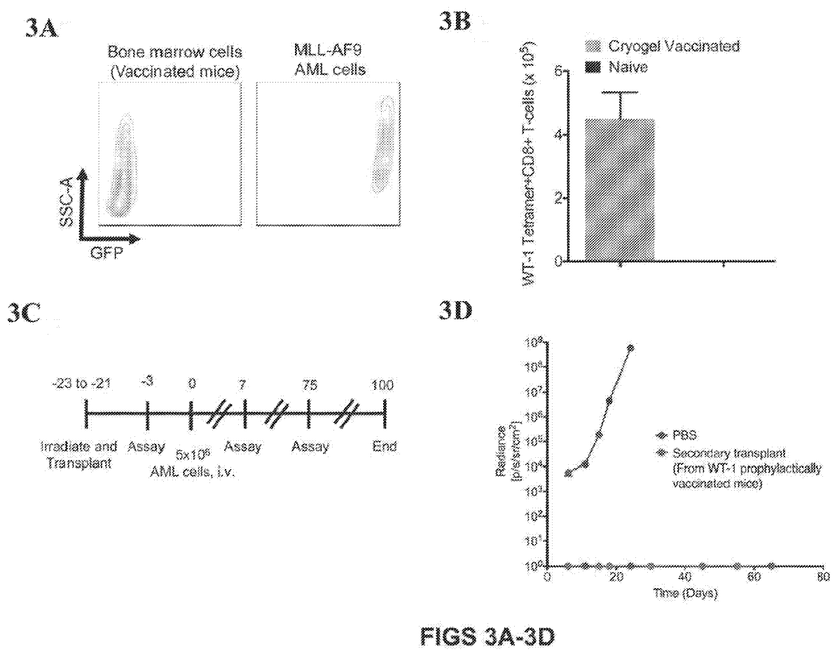

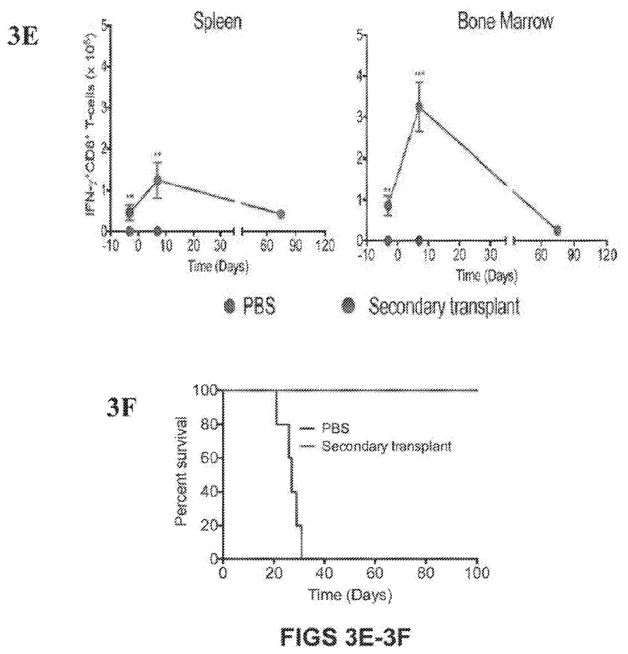

To determine whether there were residual AML cells in the vaccinated mice, bone marrow was harvested and pooled 150 days after the primary AML challenge from all mice vaccinated with the cryogel vaccine containing WT-1₁₂₆₋₁₃₄ as the antigen. The absence of GFP expressing cells in the harvested bone marrow suggested a lack of residual AML (FIG. 3A). WT-1 tetramer⁺ CD8⁺ T-cells were present in the bone marrow of the vaccinated mice, in contrast to an absence of cells in pooled bone marrow naïve controls (FIG. 3B). To determine whether (i) there was leukemia initiating potential in the harvested cells and (ii) cells from immunized mice could be adoptively transferred, secondary bone marrow transplants were performed (FIG. 3C). Recipient mice of the same C57Bl/6 genetic background as the vaccinated mice were conditioned and injected intravenously with 5 million pooled bone marrow cells from the vaccinated donors. The recipient mice did not develop AML (FIG. 3D). To test whether bone marrow transplantation conferred functional immune protection against AML, transplanted mice were then challenged with 5 million MLL-AF9 AML cells 14 days after transplantation. Substantial numbers of IFN-γ⁺CD8⁺ T cells were measured in the spleen and bone marrow of the transplanted mice as compared to control mice, which did not receive the transplant (FIG. 3E). The response in mice transplanted with bone marrow from vaccinated donors recapitulated the dynamics of the vaccinated mice, as all mice transplanted with bone marrow from vaccinated donors survived the challenge (FIG. 3F). Mice that received just control intravenous injection of phosphate buffer saline (PBS) succumbed to AML between days 23 and 26.

Induction Chemotherapy and Therapeutic Vaccination Prevents AML Relapse in Established Disease

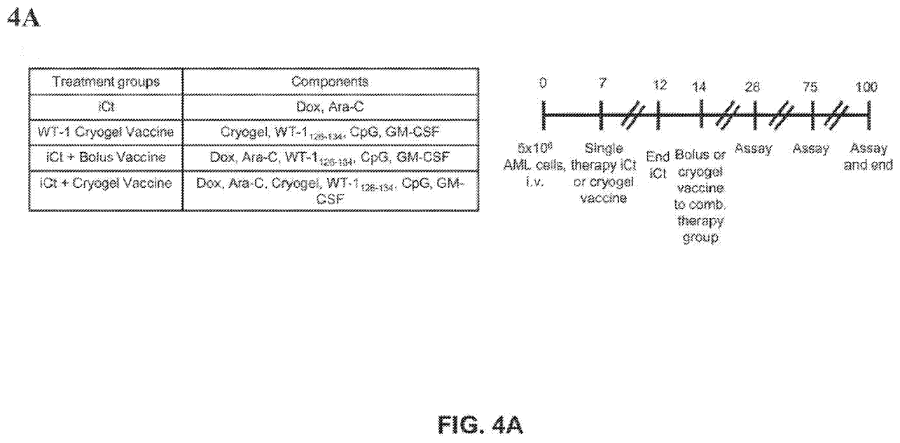

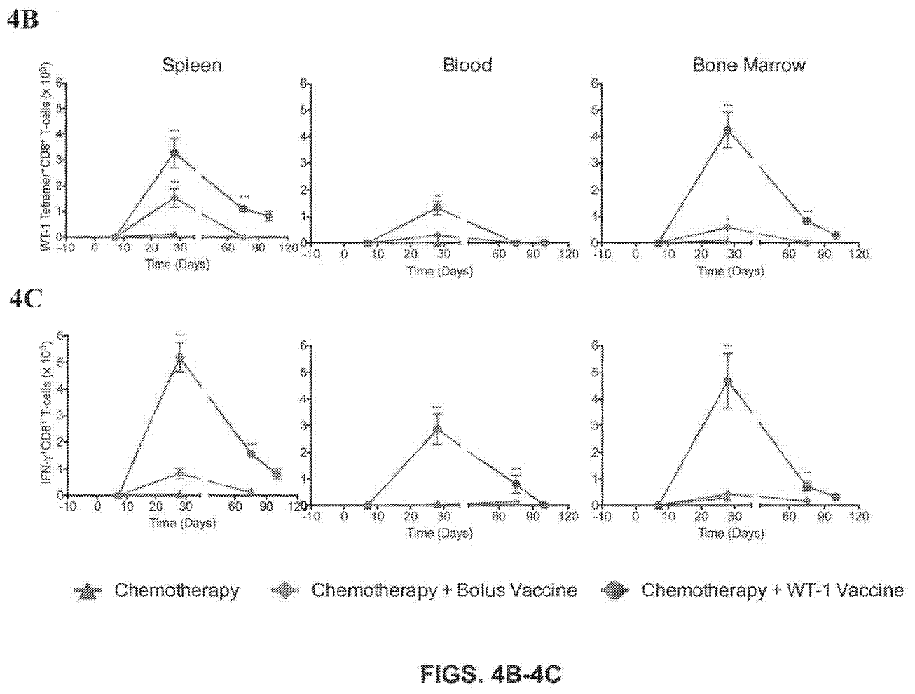

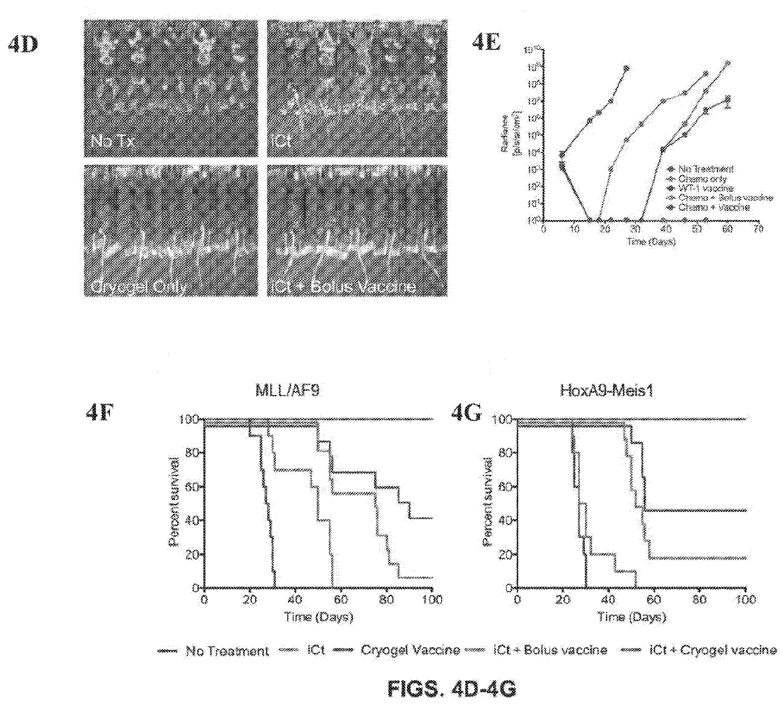

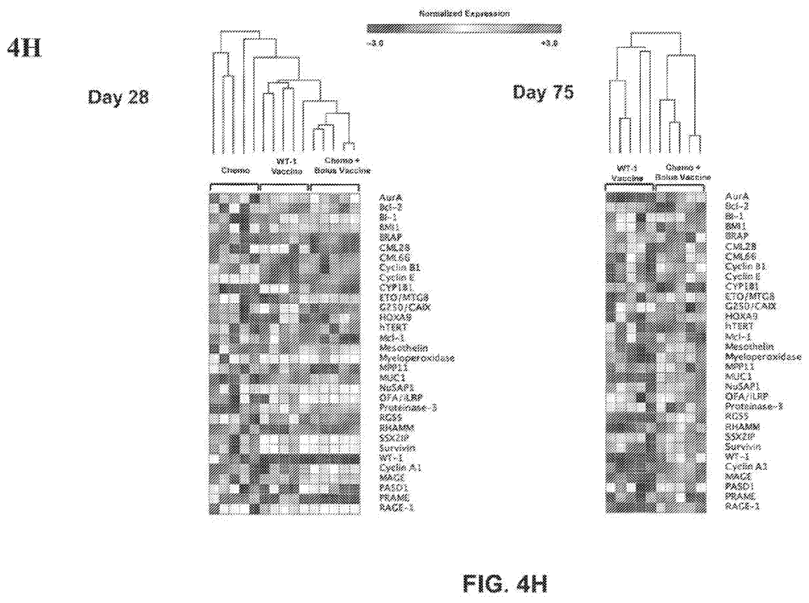

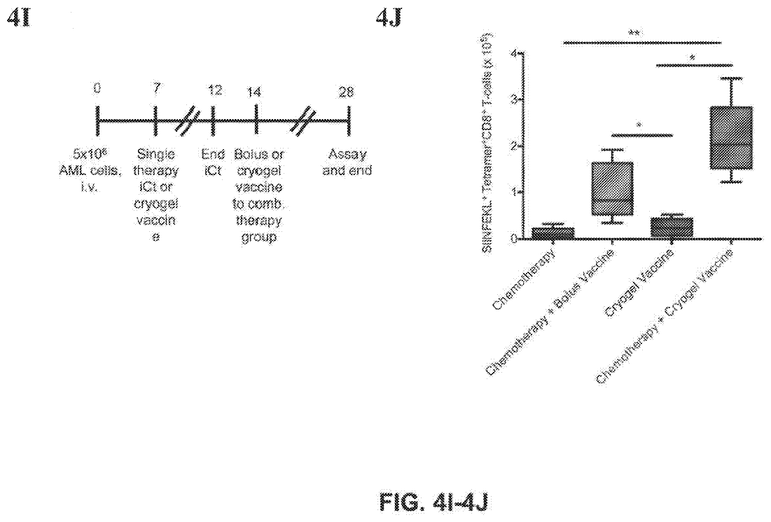

The bolus and cryogel vaccine containing WT-1₁₂₆₋₁₃₄ was tested in combination with a cytotoxic induction chemotherapy (iCt) regimen. The iCt consisted of a combination of doxorubicin hydrochloride (Dox, 3 mg/kg) and cytarabine (cytosine arabinoside, Ara-C, 100 mg/kg), administered via intraperitoneal injection every day for 3 and 5 days, respectively. The treatment duration followed the standard protocol for iCt for established acute myeloid leukemia in mice (27). Mice were injected intravenously with 5 million AML cells (FIG. 4A) and at 7 days after inoculation the presence of AML cells was confirmed using bioluminescence. AML engraftment was consistent in all animals and detected primarily in the region of the tibia and femur (>90% total bioluminescence; FIG. 4B). Subsequently, mice were divided into the following groups: (i) no treatment, (ii) iCt, (iii) cryogel vaccine, (iv) iCt and bolus vaccine or (v) iCt and cryogel vaccine. Two days after administration of the final dose of cytarabine, one group received a bolus vaccine and another group received the cryogel vaccine.

To investigate the differences in the antigen-specific response in the iCt treated groups, the frequency of WT-1 tetramer⁺ CD8⁺ T-cells and IFN-γ⁺ CD8⁺ T-cells from the blood, spleen and bone marrow were analyzed (FIG. 4B-C). The iCt alone resulted in very low levels (<5000 cells) of short-lived IFN-γ⁺CD8⁺ T-cell response and no detectable RMFPNAPYL (SEQ ID NO: 1) tetramer⁺ CD8⁺ T-cells in the hematopoietic compartments. However, when iCt was combined with either bolus vaccination or cryogel vaccination, the IFN-γ+ and RMFPNAPYL (SEQ ID NO: 1) tetramer⁺ CD8⁺ T-cell responses were significantly higher at day 28 relative to iCt alone (ANOVA with a Tukey post hoc test; n=5 per group per time point). At day 28, the magnitude of the cryogel vaccine response in the spleen was 6.4-fold and 2.1-fold higher than bolus vaccination in regards to IFN-γ⁺CD8⁺ T-cells and RMFPNAPYL (SEQ ID NO: 1) tetramer⁺ CD8⁺ T-cells respectively.

The GFP-luciferase bioluminescence reporter was used to measure leukemia burden (FIG. 4D, E). The signal increased exponentially in untreated mice, whereas the leukemia reduced significantly after mice were treated with either the iCt or the WT-1 vaccine alone. However, the AML relapsed in mice treated with iCt alone, between day 14 and day 21 and subsequently increased exponentially. The vaccine alone and the iCt with the bolus vaccine suppressed AML growth for at least 1 month after the initial AML challenge. However, the AML relapsed at about the same time in both these groups and increased exponentially but at a significantly slower rate in the cryogel vaccinated mice. The AML did not relapse in any of the mice that received combination iCt and cryogel vaccine. Survival of the mice corresponded to bioluminescence and leukemia burden (FIG. 4F): iCt alone increased survival, which was enhanced by the co-administration of the bolus vaccine. The cryogel vaccine alone prolonged survival, and when relapse occurred the mice succumbed at a slower rate than mice that received both the iCt and the bolus vaccine (log-rank test; n=10 per group). All mice that received the iCt and cryogel vaccine survived (FIG. 4F).

To Determine if the Therapeutic Benefits Extended to Other Models of AML, we tested the treatment regimens in a GFP-luciferase expressing HoxA9-Meis1 AML model. This model had a similar rate of aggressive lethality, and the treatment followed the regimen described in FIG. 4A. The trends in the treatment groups were similar to that of the MLL-AF9 AML model, in which the combination iCt and the therapeutic vaccine regimen confer full immune protection (FIG. 4G).

Combination iCt and Cryogel Vaccine Promote De Novo T-Cell Responses

Next, we investigated the generation of de novo adaptive immune responses for AML-associated antigens that were not encoded by the vaccine. Within the treatment groups in which mortality was observed, a targeted gene expression analysis was conducted in a subset of known AML-associated antigens from AML cells isolated from terminally ill mice on Days 28 and Day 75 (FIG. 4H). At the Day 28 time-point, the iCt+bolus vaccine treatment was broadly suppressive of AML-antigens relative to the WT-1 cryogel vaccine or iCt alone. At Day 75, the WT-1 cryogel vaccine treated mice had an overall lower relative expression of AML associated antigens, relative to the iCt+bolus vaccine treatment. To determine if the protection conferred by the cryogel vaccine was mediated by an adaptive immune response that extended beyond WT-1, mice were inoculated with ovalbumin (OVA)—expressing AML cells (oAML). Mice then received: (i) iCt, (ii) cryogel vaccine containing WT-1₁₂₆₋₁₃₄ as the antigen, (iii) iCt and cryogel vaccine containing WT-1₁₂₆₋₁₃₄ as the antigen (FIG. 4I). Twenty eight days after inoculation with the oAML cells, the number of SIINFEKL (SEQ ID NO: 2) tetramer⁺ CD8⁺ T-cells were significantly higher in the mice which received both iCt and the cryogel vaccine (FIG. 4J; ANOVA with a Tukey post hoc test; n=5 per group per time point). While iCt alone resulted in a weak response, the iCt+bolus vaccine generated significantly higher numbers of SIINFEKL (SEQ ID NO: 2) tetramer⁺ CD8⁺ T-cells, when compared to both the iCt or cryogel vaccine alone (ANOVA with a Tukey post hoc test; n=5 per group per time point).

Combination iCt and Cryogel Vaccination Deplete Leukemia Initiating Cells

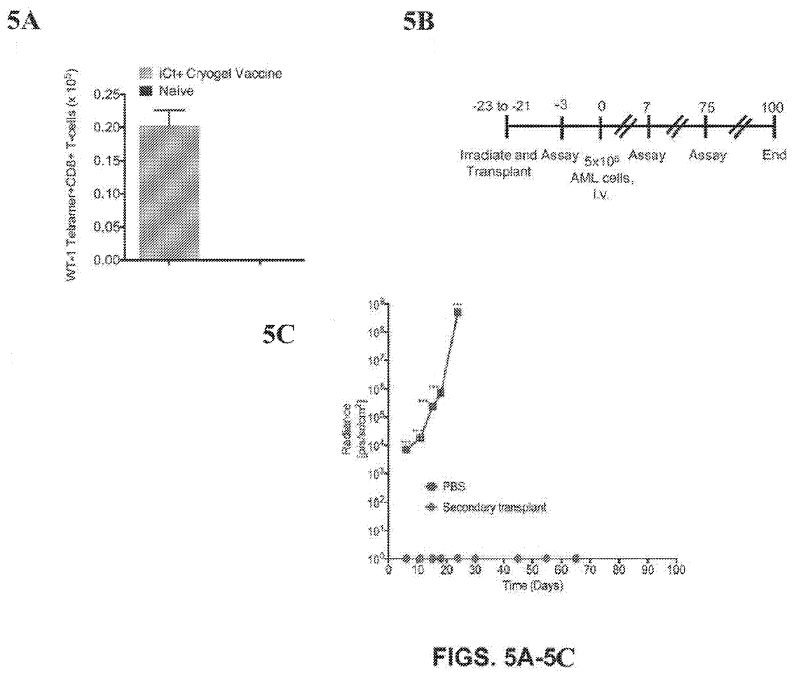

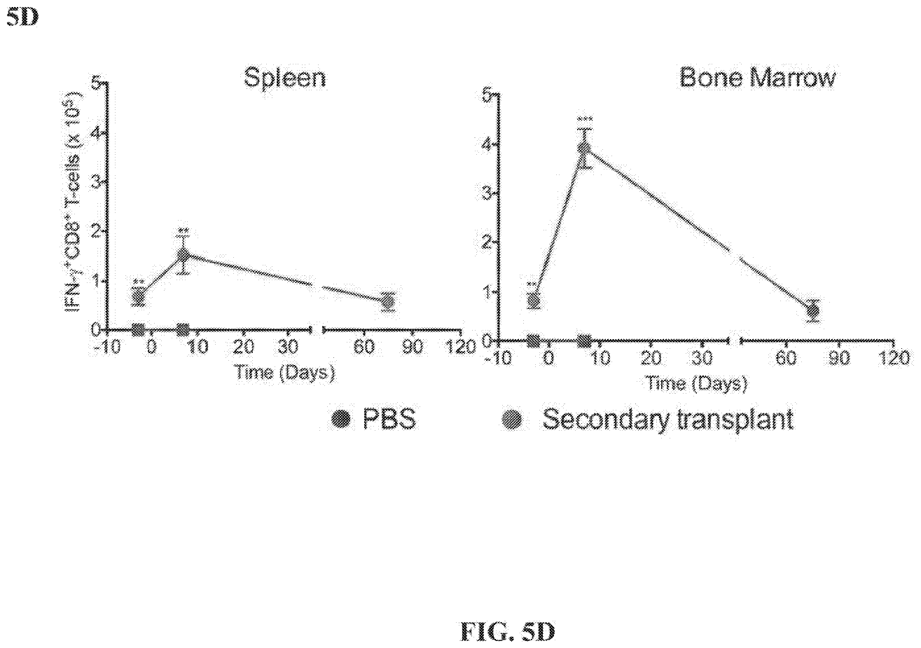

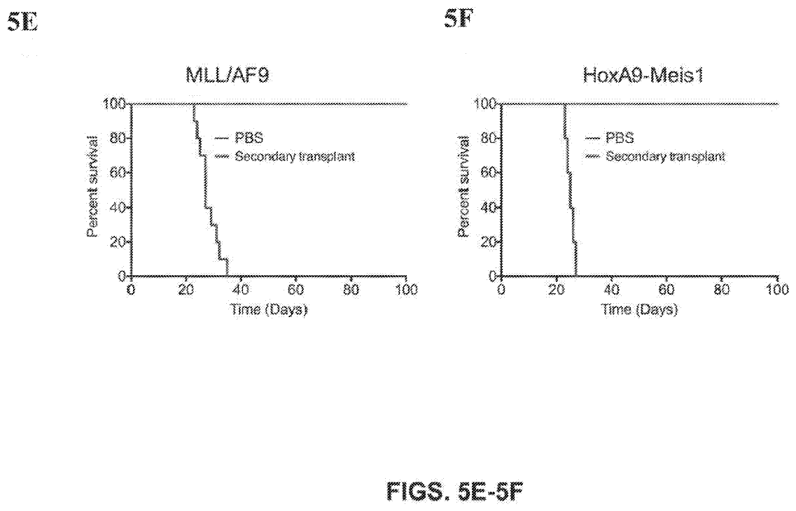

To determine the presence of residual leukemia initiating cells in the mice that received the iCt and cryogel vaccine containing WT-1₁₂₆₋₁₃₄ as the antigen, bone marrow was harvested from treated mice at Day 100 after the initial MLL-AF9 AML challenge. Higher levels of RMFPNAPYL (SEQ ID NO: 1) tetramer⁺ CD8⁺ T-cells were observed in the bone marrow of the vaccinated mice, compared with naïve controls (FIG. 5A, ANOVA with a Tukey post hoc test; n=5 per group per time point). Secondary bone marrow transplants were subsequently performed (FIG. 5B), in which C57Bl/6 mice were injected intravenously with 5 million pooled bone marrow cells from the vaccinated donors and periodically imaged using bioluminescence imaging (FIG. 5C). After confirming that the recipient mice did not develop AML over 14 days, transplanted mice were challenged with 5 million MLL-AF9 AML cells to test for functional immune protection 14 days after transplantation. IFN-γ⁺ CD8⁺ T-cells were measured in the spleen and bone marrow of the transplanted mice (FIG. 5D). The response in mice transplanted with bone marrow from vaccinated donors recapitulated the dynamics of the vaccinated mice. All transplanted mice survived the challenge (FIG. 5E). Mice that received just control intravenous injection of phosphate buffer saline (PBS) succumbed to AML between days 26 and 31 as expected. Similarly, secondary transplantation using bone marrow from surviving mice challenged with the HoxA9-Meis1 AML cells did not result in manifestation of AML in the transplanted mice and mice were able to overcome a challenge (FIG. 5F).

### DISCUSSION

Patients with AML present with a high burden of disseminated disease at the time of diagnosis and require effective and tolerable systemic therapy. Chemotherapy can induce an apparent remission but relapse occurs in the majority of patients, highlighting the difficulty in eradicating all AML cells. Therapeutic vaccines have the potential of achieving a lasting AML-specific immune response capable of eradicating the residual disease that remains following chemotherapy. The development of clinically relevant cancer vaccines requires T-cell activation resulting from effective presentation of tumor antigen in the context of co-stimulation (28). This study demonstrates that an injectable cryogel vaccine can create a local, controlled immunological microenvironment and serve as a site for regulation of the immune response against AML. The cryogel vaccines locally deliver immunoregulatory factors and AML-associated antigen WT-1₁₂₆₋₁₃₄ to evoke a potent and durable response against AML. In a model of established AML, the cryogel vaccine alone extends survival, and when used in combination with induction chemotherapy, eradicates the disease.

The mode and delivery mechanism of an AML vaccine is key to its efficacy. It has been demonstrated that the requirements for multiple vaccinations for efficacy can significantly down regulate the cytotoxic T-cell response (29-31). Extended release antigen/adjuvant delivery strategies such as water-in-oil emulsions can release for several months but may lead to a deficient immune response at the site of the disease (32). The vaccine cryogel is a single subcutaneous injection that elicited a robust immune response that had efficacy against AML in both a prophylactic and therapeutic setting. We did not observe a deficiency in the long-term cell-mediated immune response with the cryogel vaccine, as evidenced by the eradication of the leukemia and efficacy in preventing AML engraftment after secondary transplantation in mice.

The prophylactic administration of the cryogel vaccine elicited a strong and durable systemic immune response, compared with the bolus vaccine. We observed that the induction of a WT-1 specific CTL response by the vaccine cryogel induced cell lysis in an AML-specific manner as measured by a thymidine-release assay in vitro. The cryogel vaccinated mice rejected the engraftment of AML cells after the primary AML challenge, with both AML cell lysates and the WT-1₁₂₆₋₁₃₄ peptide serving as effective vaccine antigens. Moreover mice were able to overcome a re-challenge after 100 days, indicating the potential of these vaccines to establish a long-term immunity. The induction of these strong cellular immune responses is likely a result of the high number of dendritic cells, their sustained and prolonged activation and priming, and their subsequent interactions with immune cells in the lymph node. In contrast to some DC adoptive transfer techniques for prophylactic AML vaccination, efficacy is observed without the need for pre-conditioning lymphodepletion regimens to deplete immunosuppressive cells(33, 34). Importantly, the identification of AML-associated antigens has created interest in opportunities where at-risk individuals can be identified in advance and prophylactically immunized against AML. The cryogel vaccine platform is also well suited to be combined with sequencing of patient tumors for neoantigen identification to personalize the vaccine, and to explore potential synergies with T-cell and other adoptive transfer techniques(35-38).

The benefit in overall survival with cryogel vaccination is most notably observed in the murine models of established AML. The iCt reduced the leukemia bulk, which was a logical prelude to administering the cryogel vaccine to eradicate residual disease and prevent relapse. Mice treated with iCt and the cryogel vaccine also demonstrated responses to additional antigens not present in the vaccine. Although it remains an area of active investigation, some previous studies have indicated the potential immunostimulatory capacity of anthracylines, such as doxorubicin, in treating AML(39, 40). The ability of the combination therapy to promote antigen spread likely protected against cells lacking the vaccine-targeted antigens in our study. Taken together, the results from the single therapy cryogel vaccine and the combination iCt and cryogel vaccine therapy suggest that the latter is important for dealing with AML heterogeneity and preventing immune escape. In contrast to AML vaccine clinical trials, which have focused on preventing AML relapse in patients who are months to years into a clinical remission, the results from our study suggest that the major benefit of deploying the cryogel vaccine may be found in AML patients immediately after chemotherapy(41).

The cryogel vaccine conferred protection against AML and also resulted in eradication of leukemia initiating cells, as indicated by the failure of AML to manifest after secondary transplant in recipients. Prior studies have demonstrated that there can be a selective elimination of leukemic initiating cells, but not normal progenitors, by WT1-specific cytotoxic CD8⁺ T cells(42). Furthermore, the transplant provided lasting immunological protection given that all transplanted mice survived an AML re-challenge. The results suggest that both cell-mediated as well as humoral immunity contribute to the targeted destruction of AML cells.

The cryogel vaccine treatment was well-tolerated and promoted AML rejection without the indication of pancytopenia or autoimmunity in the studies. While targeting cancer-associated antigens may carry the risk of autoimmunity, it has been demonstrated that the long-term presence of WT-1-specific T-cells does not result in the development of autoimmunity(43). Similarly, although antigen spreading as observed in this study may promote AML rejection, an issue for future work is to understand the contributions of the humoral immunity and whether the de novo immune responses are focused on AML-associated antigens or on self-antigens in the long-term. In the clinic, the importance of maintaining a balance between appropriate immunological activation while preventing an over-exuberant reaction is well known following the treatment of AML patients with HSCT, in which the GvL effects are associated with the graft-versus-host disease. Clinical studies have observed that a rapid expansion of pre-existing lymphocytes in the HSCT graft promotes T-cell reactivity and donor-derived T-cells protect against relapse(44). Since HSCT is currently used as a treatment for AML in most eligible patients, it would be interesting to explore the use of the cryogel vaccine after HSCT, which may have efficacy against AML (45, 46). Another area of application could be the combination of lower intensity iCt and the cryogel vaccination treatment, which could be applicable in older patients who constitute the bulk of AML patients but experience particularly poor outcomes as they are unable to undergo HSCT(47).

We have demonstrated that a biomaterial-based cryogel vaccine targeting a defined antigen can lead to robust immune responses against AML. The prevention of AML engraftment relied on a prophylactic vaccination strategy to activate cell-mediate immunity against AML, whereas the eradication of established AML relied on a broader response, elicited by combining iCt and the cryogel vaccine. Our findings suggest that induction of a specific anti-leukemia immune response in AML patients during a period of remission or minimal residual disease might prevent the life-threatening evolution of this disease.

Methods

Rationale and Study Design

The role of an injectable biomaterial-based cryogel cancer vaccine in treating AML was investigated in a murine model. The in vitro sustained release of the vaccine components, cytokine GM-CSF, antigen and adjuvant, was measured using protein assays. In vivo, the concentration of relevant subpopulations of immune cells at the site of the vaccine and the lymph nodes were quantified at different time points using flow cytometry. Mice were (i) prophylactically vaccinated and subsequently challenged with acute myeloid leukemia and (ii) inoculated with leukemia and vaccinated following chemotherapy. The development of leukemia-specific immune response was used to assess vaccine potency. The quantification of leukemia burden and survival were used to assess disease progression and the efficacy of the therapy. Secondary transplantation was used to test for residual leukemia and transfer of immunity. The sample size for the experiments were chosen based on estimates from pilot experiments and previously published results such that appropriate statistical tests could yield significant results. All animal experiments were conducted with n≥4 per group and all survival experiments were conducted with n≥10 per group to fulfill the minimum requirement for nonparametric statistical analysis. Survival experiments were repeated at least twice.

Materials

UP LVG sodium alginate with high guluronate content was purchased from ProNova Biomedical; 2-morpholinoethanesulfonic acid (MES), sodium chloride (NaCl), sodium hydroxide (NaOH), N-hydroxysuccinimide (NHS), 1-ethyl-3-(3-dimethylaminopropyl)-carbodiimide hydrochloride (EDC), 2-aminoethyl methacrylate hydrochloride (AEMA) and acetone were purchased from Sigma-Aldrich. ACRL-PEG-NHS (3.5 kDa) and 4arm PEG Acrylate (10 kDa) were purchased from JenKem Technology. Animals used in this study were, C57BL/6 (Jackson Laboratories). All animals were female and between 6 and 8 weeks old at the start of the experiment. Syngeneic murine HoxA9-Meis1 and MLL/AF9 leukemia cell lines were previously generated and used as described (48). The WT-1 peptide RMFPNAPYL (Arg-Met-Phe-Pro-Asn-Ala-Pro-Tyr-Leu) (SEQ ID NO: 1) was custom made by Peptide 2.0.

Cryogel Vaccine Fabrication

The cryogel vaccine was made following a previously described technique with some modifications (21). Methacrylated alginate (MA-alginate) was prepared by reacting alginate with AEMA. Sodium alginate was dissolved in a buffer solution (0.6% (wt/vol), pH ˜6.5) of 100 mM MES buffer. NHS and EDC were added to the mixture to activate the carboxylic acid groups on the alginate backbone followed by AEMA (molar ratio of NHS:EDC:AEMA=1:1.3:1.1) and the solution was stirred at room temperature (RT) for 24 h. The mixture was precipitated in acetone, filtered and dried in a vacuum oven overnight at RT. Alginate-PEG cryogel vaccines were synthesized by preparing a 2.5 wt % solution of MA-alginate and 4arm PEG Acrylate macromonomers (molar ratio MA-alginate: 4arm PEG Acrylate=4:1) in deionized water and subsequently adding tetramethylethylenediamine (TEMED) (0.5% (wt/vol)) and ammonium persulfate (APS) (0.25% (wt/vol)). CpG ODN 1826, 5′-TCC ATG ACG TTC CTG ACG TT-3′ (Invivogen), and GM-CSF (PeproTech) and the antigen (lysate or peptide) were added to the polymer solution before cryopolymerization. All precursor solution was precooled to 4° C. to decrease the rate of polymerization before freezing. After addition of the initiator to the prepolymer solution, the solution was quickly transferred onto a precooled (−20° C.) Teflon mold. After overnight incubation, the gels were thawed and collected in petri dishes on ice.

Biomolecule Release Quantification

To determine the incorporation efficiency and release kinetics of CpG ODN, GM-CSF and antigen from cryogel vaccines, gels were incubated in 1 ml of sterile PBS at 37° C. with shaking. Media was replaced periodically. Micro-BCA (Pierce Biotechnology) was used to quantify total protein content. GM-CSF and CpG ODN released in the supernatant were detected by ELISA (Invitrogen) and OliGreen assay (Invitrogen), respectively. The amount of antigen was determined by subtracting total protein content from the amount of GM-CSF quantified by ELISA.

In Vivo Cryogel Vaccine Delivery and Cell Trafficking

All animal work was approved by the Harvard Institutional Animal Care and Use Committee and in followed the National Institutes of Health guidelines. Female C57BL/6 mice (Jackson Laboratory), 6-8 weeks of age, were anaesthetized and received subcutaneous injections of two cryogels or bolus vaccines, which were suspended in 0.2 ml of sterile PBS, into the dorsal flank by means of a 16-gauge needle. One cryogel was injected on each side of the spine and positioned approximately midway between the hind and fore-limbs. Subcutaneous nodule size was quantified over time by measuring the nodule length, width and height using a caliper. To quantify and characterize cell infiltrates at the site of the vaccine, cryogels were harvested from euthanized mice at pre-determined time intervals, cut into smaller pieces and digested with collagenase/dispase (˜250 U ml⁻¹; Roche) at 37° C. for 30 min under agitation. The suspensions were passed through a 40-μm cell strainer to reduce scaffold particles. The cells were counted and assessed for viability with a Cellometer (Nexcelom). The draining lymph nodes were harvested and suspensions from dLNs were prepared by mechanical disruption and pressing of the tissue against 40-μm cell strainers, and single cells were prepared for analysis.

Prophylactic and Therapeutic Vaccine Study

Animals were immunized with 2 cryogel vaccines (˜30 μl each) containing either cell lysates or WT-1₁₂₆₋₁₃₄ peptide as the antigen, and bolus vaccine containing 100 μg peptide, 100 μg CpG-ODN and 1 μg GM-CSF. After 10 days, animals were challenged with an intravenous injection of 5×10⁶ MLL/AF9 AML cells and leukemia progression was monitored. After 100 days, the surviving mice were re-challenged with 5×10⁶ MLL/AF9 leukemia cells. For mice challenged with either 5×10⁶ MLL/AF9 or HoxA9-Meis1 AML cells, induction chemotherapy or the cryogel vaccine was administered 7 days after challenge and consisted of 100 mg/kg cytarabine (Ara-C) for 5 days and 3 mg/kg doxorubicin for 3 days. Cryogel vaccines contained WT-1₁₂₆₋₁₃₄ peptide as the antigen. Leukemia burden was monitored by bioluminescence imaging. At pre-determined time intervals, blood, bone marrow and the spleen were collected from euthanized mice in the vaccination studies. Bone marrow was collected by crushing the tibia, femur and pelvis. Splenocytes were isolated by mechanical disruption of the spleen against 40-μm cell strainers. Red blood cells in the harvested tissues were lysed using ACK Lysing buffer (Lonza) and leukocytes were prepared for analysis.

Flow Cytometry Analysis

Antibodies to CD8-α (53-6.7), IFN-γ (XMG1.2), CD3-ε (145-2C11), B220 (RA3-6B2), Ly-6G (1A8), F4/80 (BM8), CD11b (M1/70), CD11c (N418), CD14 (Sa14-2) and CD86 (GL-1) were purchased from BioLegend. WT-1 tetramer (Alexa Fluor 647 H-2Kd RMFPNAPYL (SEQ ID NO: 1)) and SIINFEKL (SEQ ID NO: 2) tetramer (Alexa Fluor 647 H-2Kb OVA) were obtained from the NIH Tetramer Core Facility. Intracellular cytokine staining of IFN-γ was performed using Fixation and Permeabilization Solution Golgiplug (BD Biosciences) following the manufacturer's protocol. Peptides used for re-stimulation were 10 μg/ml of the relevant antigen. All cells were gated based on forward and side-scatter characteristics to limit debris including dead cells. Antibodies were diluted according to the manufacturer's suggestions. Cells were gated based on positive controls, and the percentages of cells staining positive for each marker was recorded.

DNA Fragmentation Assay for Measuring WT-1 Specific CTL Activity

The spleens of the animals were isolated and gently homogenized at day 10 after prophylactic vaccination. CD8+ T cells were magnetically sorted from each spleen (Miltenyi Biotec). The T cells were then co-cultured with LPS (100 ng/ml)-primed bone marrow derived dendritic cells pulsed with 1 μM WT-1 peptide for 24 h in round-bottomed, 96-well plates. CD8+ T cells and dendritic cells were co-cultured at the ratio of 2 to 1 (T to dendritic cell). Following induction of the WT-1 specific CTLs, thymidine release from killed target AML cells (described previously (49)), was used to assess in vitro CTL activity. Briefly, target AML cells are labeled with [3H]thymidine and mixed with cytotoxic effector cells, isolated from the spleen of cryogel vaccinated or naïve mice. The percent lysis was calculated by comparing the amount of [3H]thymidine labeled DNA fragments in the presence and absence of effector cells.

Transplant for Leukemia-Initiating Cell Analysis

Bone marrow cells from treated mice and control wild-type mice were isolated by harvesting, crushing and pooling cells from the femur, pelvis and tibia. 5×106 live cells from either treated or control wild-type mice were injected into recipient mice without conditioning.

Gene Expression Analysis

GFP-expressing cells were isolated from the bone marrow using fluorescence activated cell sorting. Total RNA was isolated from using QIAGEN RNeasy-Plus Mini columns, with additional on-column DNase treatment to eliminate traces of genomic DNA. cDNA was synthesized with a high-capacity cDNA archive kit (Applied Biosystems; ABI). Equal volumes of cDNA and TaqMan Universal PCR Master Mix (ABI) were combined and loaded into the ports of TaqMan custom low-density arrays following the manufacturer's instructions. Real-time PCR was performed on StepOnePlus Real-Time PCR System (ABI).

Statistical Analysis

Experiments were performed by at least two researchers and were not blinded. Results were analyzed by using one-way ANOVA with a Tukey post hoc test using GraphPad Prism software. Where ANOVA was used, variance between groups was found to be similar by Bartlett's test. Survival curves were analyzed by using the log-rank (Mantel-Cox) test. No samples were excluded from analysis.

### REFERENCES

15. D. J. Irvine, Materializing the future of vaccines and immunotherapy. Nature Reviews Materials 1, 15008 (2016).
16. L. Gu, D. J. Mooney, Biomaterials and emerging anticancer therapeutics: engineering the microenvironment. Nature Reviews Cancer 16, 56-66 (2016).
17. J. L. Zakrzewski, M. R. Van Den Brink, J. A. Hubbell, Overcoming immunological barriers in regenerative medicine. Nature biotechnology 32, 786 (2014).
18. K. D. Moynihan, C. F. Opel, G. L. Szeto, A. Tzeng, E. F. Zhu, J. M. Engreitz, R. T. Williams, K. Rakhra, M. H. Zhang, A. M. Rothschilds, Eradication of large established tumors in mice by combination immunotherapy that engages innate and adaptive immune responses. Nature Medicine, (2016).
19. J. Leleux, K. Roy, Micro and nanoparticle-based delivery systems for vaccine immunotherapy: an immunological and materials perspective. Advanced healthcare materials 2, 72-94 (2013).
20. J. S. Rudra, Y. F. Tian, J. P. Jung, J. H. Collier, A self-assembling peptide acting as an immune adjuvant. Proceedings of the National Academy of Sciences 107, 622-627 (2010).
21. S. A. Bencherif, R. Warren Sands, O. A. Ali, W. A. Li, S. A. Lewin, T. M. Braschler, T. Y. Shih, C. S. Verbeke, D. Bhatta, G. Dranoff, D. J. Mooney, Injectable cryogel-based whole-cell cancer vaccines. Nature communications 6, 7556 (2015); published online EpubAug 12 (10.1038/ncomms8556).
22. J. Kim, W. A. Li, Y. Choi, S. A. Lewin, C. S. Verbeke, G. Dranoff, D. J. Mooney, Injectable, spontaneously assembling, inorganic scaffolds modulate immune cells in vivo and increase vaccine efficacy. Nat Biotechnol 33, 64-72 (2015); published online EpubJan (10.1038/nbt.3071).
23. O. A. All, N. Huebsch, L. Cao, G. Dranoff, D. J. Mooney, Infection-mimicking materials to program dendritic cells in situ. Nat Mater 8, 151-158 (2009); published online EpubFeb (10.1038/nmat2357).
24. O. A. Ali, D. Emerich, G. Dranoff, D. J. Mooney, In situ regulation of DC subsets and T cells mediates tumor regression in mice. Science translational medicine 1, 8ra19 (2009); published online EpubNov 25 (10.1126/scitranslmed.3000359).
25. G. J. Randolph, J. Ochando, S. Partida-Sánchez, Migration of dendritic cell subsets and their precursors. Annu. Rev. Immunol. 26, 293-316 (2008).
26. M.-C. Dieu, B. Vanbervliet, A. Vicari, J.-M. Bridon, E. Oldham, S. Aït-Yahia, F. Brière, A. Zlotnik, S. Lebecque, C. Caux, Selective recruitment of immature and mature dendritic cells by distinct chemokines expressed in different anatomic sites. The Journal of experimental medicine 188, 373-386 (1998).
27. J. Zuber, I. Radtke, T. S. Pardee, Z. Zhao, A. R. Rappaport, W. Luo, M. E. McCurrach, M. M. Yang, M. E. Dolan, S. C. Kogan, J. R. Downing, S. W. Lowe, Mouse models of human AML accurately predict chemotherapy response. Genes & development 23, 877-889 (2009); published online EpubApr 1 (10.1101/gad. 1771409).
28. K. Palucka, J. Banchereau, Cancer immunotherapy via dendritic cells. Nature reviews. Cancer 12, 265-277 (2012); published online EpubMar 22 (10.1038/nrc3258).
29. K. Rezvani, A. S. Yong, S. Mielke, B. Jafarpour, B. N. Savani, R. Q. Le, R. Eniafe, L. Musse, C. Boss, R. Kurlander, Repeated PR1 and WT1 peptide vaccination in Montanide-adjuvant fails to induce sustained high-avidity, epitope-specific CD8+ T cells in myeloid malignancies. Haematologica 96, 432-440 (2011).
30. I. Melero, G. Gaudernack, W. Gerritsen, C. Huber, G. Parmiani, S. Scholl, N. Thatcher, J. Wagstaff, C. Zielinski, I. Faulkner, Therapeutic vaccines for cancer: an overview of clinical trials. Nature reviews Clinical oncology 11, 509-524 (2014).
31. Y. Hailemichael, Z. Dai, N. Jaffarzad, Y. Ye, M. A. Medina, X.-F. Huang, S. M. Dorta-Estremera, N. R. Greeley, G. Nitti, W. Peng, Persistent antigen at vaccination sites induces tumor-specific CD8+ T cell sequestration, dysfunction and deletion. Nature medicine 19, 465-472 (2013).
32. W. Herbert, The mode of action of mineral-oil emulsion adjuvants on antibody production in mice. Immunology 14, 301 (1968).
33. S. Delluc, P. Hachem, S. Rusakiewicz, A. Gaston, C. Marchiol-Fournigault, L. Tourneur, N. Babchia, D. Fradelizi, A. Regnault, K. H. L. Q. Sang, Dramatic efficacy improvement of a DC-based vaccine against AML by CD25 T cell depletion allowing the induction of a long-lasting T cell response. Cancer immunology, immunotherapy 58, 1669-1677 (2009).
34. L. Gattinoni, D. J. Powell, S. A. Rosenberg, N. P. Restifo, Adoptive immunotherapy for cancer: building on success. Nature Reviews Immunology 6, 383-393 (2006).
35. D. S. Ritchie, P. J. Neeson, A. Khot, S. Peinert, T. Tai, K. Tainton, K. Chen, M. Shin, D. M. Wall, D. Hnemann, Persistence and efficacy of second generation CAR T cell against the LeY antigen in acute myeloid leukemia. Molecular Therapy 21, 2122-2129 (2013).
36. M. L. Davila, D. C. Bouhassira, J. H. Park, K. J. Curran, E. L. Smith, H. J. Pegram, R. Brentjens, Chimeric antigen receptors for the adoptive T cell therapy of hematologic malignancies. International journal of hematology 99, 361-371 (2014).
37. M. M. Gubin, M. N. Artyomov, E. R. Mardis, R. D. Schreiber, Tumor neoantigens: building a framework for personalized cancer immunotherapy. The Journal of clinical investigation 125, 3413-3421 (2015).
38. J. Rosenblatt, R. M. Stone, L. Uhl, D. Neuberg, R. Joyce, J. D. Levine, J. Arnason, M. McMasters, K. Luptakova, S. Jain, J. I. Zwicker, A. Hamdan, V. Boussiotis, D. P. Steensma, D. J. DeAngelo, I. Galinsky, P. S. Dutt, E. Logan, M. P. Bryant, D. Stroopinsky, L. Werner, K. Palmer, M. Coll, A. Washington, L. Cole, D. Kufe, D. Avigan, Individualized vaccination of AML patients in remission is associated with induction of antileukemia immunity and prolonged remissions. Science translational medicine 8, 368ra171-368ra171 (2016)10.1126/scitranslmed.aag1298).
39. L. Zitvogel, L. Apetoh, F. Ghiringhelli, G. Kroemer, Immunological aspects of cancer chemotherapy. Nature reviews immunology 8, 59-73 (2008).
40. J. Schlom, Therapeutic cancer vaccines: current status and moving forward. Journal of the National Cancer Institute 104, 599-613 (2012).
41. L. Ding, T. J. Ley, D. E. Larson, C. A. Miller, D. C. Koboldt, J. S. Welch, J. K. Ritchey, M. A. Young, T. Lamprecht, M. D. McLellan, Clonal evolution in relapsed acute myeloid leukaemia revealed by whole-genome sequencing. Nature 481, 506-510 (2012).
42. L. Gao, S.-a. Xue, R. Hasserjian, F. Cotter, J. Kaeda, J. M. Goldman, F. Dazzi, H. J. Stauss, Human cytotoxic T lymphocytes specific for Wilms' tumor antigen-1 inhibit engraftment of leukemia-initiating stem cells in non-obese diabetic-severe combined immunodeficient recipients. Transplantation 75, 1429-1436 (2003).
43. C. Pospori, S.-A. Xue, A. Holler, C. Voisine, M. Perro, J. King, F. Fallah-Arani, B. Flutter, R. Chakraverty, H. J. Stauss, Specificity for the tumor-associated self-antigen WT1 drives the development of fully functional memory T cells in the absence of vaccination. Blood 117, 6813-6824 (2011).
44. H.-J. Kolb, Graft-versus-leukemia effects of transplantation and donor lymphocytes. Blood 112, 4371-4383 (2008).
45. V. Ho, G. Dranoff, H. Kim, M. Pasek, C. Cutler, E. Alyea, J. Koreth, J. H. Antin, R. J. Soiffer, GM-CSF Secreting Leukemia Cell Vaccinations after Allogeneic Reduced-Intensity Peripheral Blood Stem Cell Transplantation (SCT) for Advanced Myelodysplastic Syndrome (MDS) or Refractory Acute Myeloid Leukemia (AML). Blood 108, 3680-3680 (2006).
46. V. Ho, G. Dranoff, H. Kim, M. Vanneman, M. Pasek, C. Cutler, J. Koreth, E. Alyea, J. H. Antin, R. Jerome, GM-CSF Secreting Leukemia Cell Vaccination after Allogeneic Reduced Intensity Hematopoietic Stem Cell Transplantation for Advanced Myeloid Malignancies. Blood 112, 825-825 (2008).
47. (National Institutes of Health, 2016), vol. 2016.
48. D. B. Sykes, Y. S. Kfoury, F. E. Mercier, M. J. Wawer, J. M. Law, M. K. Haynes, T. A. Lewis, A. Schajnovitz, E. Jain, D. Lee, H. Meyer, K. A. Pierce, N. J. Tolliday, A. Waller, S. J. Ferrara, A. L. Eheim, D. Stoeckigt, K. L. Maxcy, J. M. Cobert, J. Bachand, B. A. Szekely, S. Mukherjee, L. A. Sklar, J. D. Kotz, C. B. Clish, R. I. Sadreyev, P. A. Clemons, A. Janzer, S. L. Schreiber, D. T. Scadden, Inhibition of Dihydroorotate Dehydrogenase Overcomes Differentiation Blockade in Acute Myeloid Leukemia. Cell 167, 171-186 e115 (2016); published online EpubSep 22 (10.1016/j.cell.2016.08.057).
49. J. Wonderlich, G. Shearer, A. Livingstone, A. Brooks, Induction and measurement of cytotoxic T lymphocyte activity. Current protocols in immunology, 3.11. 11-13.11. 23 (2006).

### Example 2

Coupling Immune Reconstitution with Vaccines for Antigen-Specific Immunity

The formation of antigen-specific CD8+ cytotoxic T-cells is key to conferring protective immunity after a HSCT. Expanding the immune repertoire after HSCT can be coupled with vaccinations against pathogens commonly associated with HSCT. As further described herein, the present inventors will vaccinate HSC transplanted mice against ovalbumin (OVA) and vaccinate against leukemia after a HSCT.

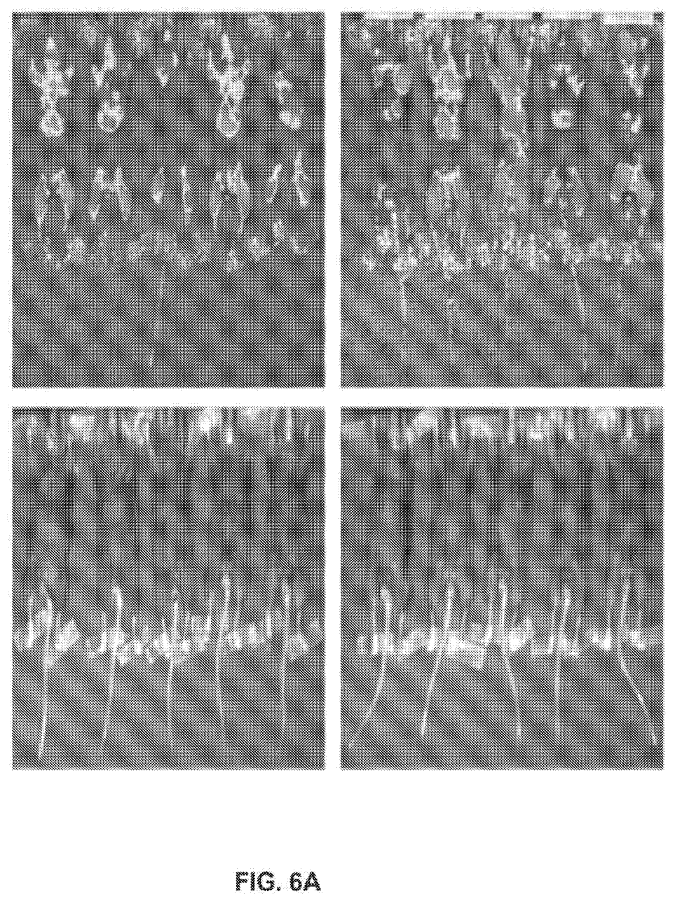

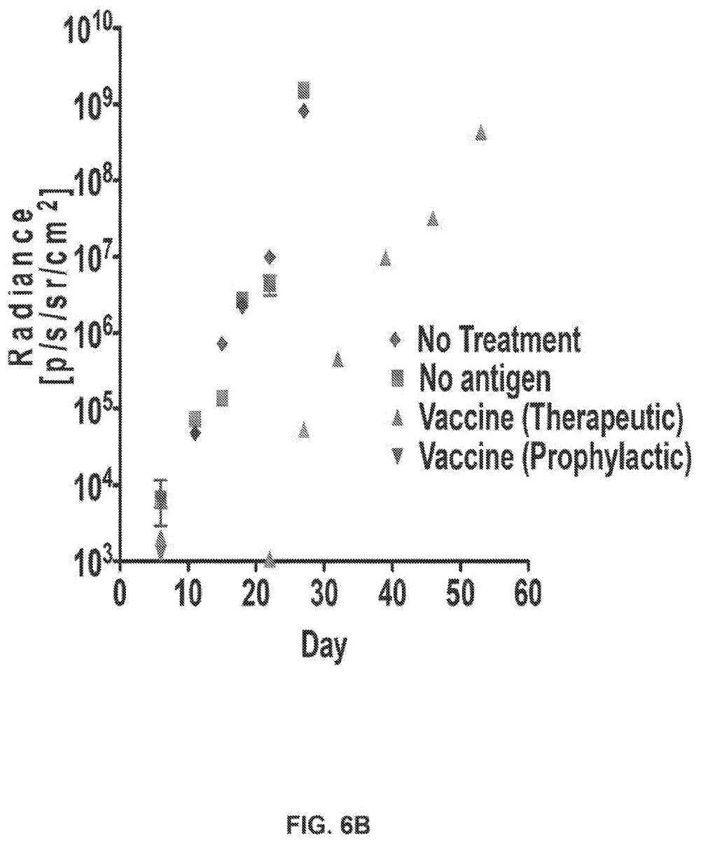

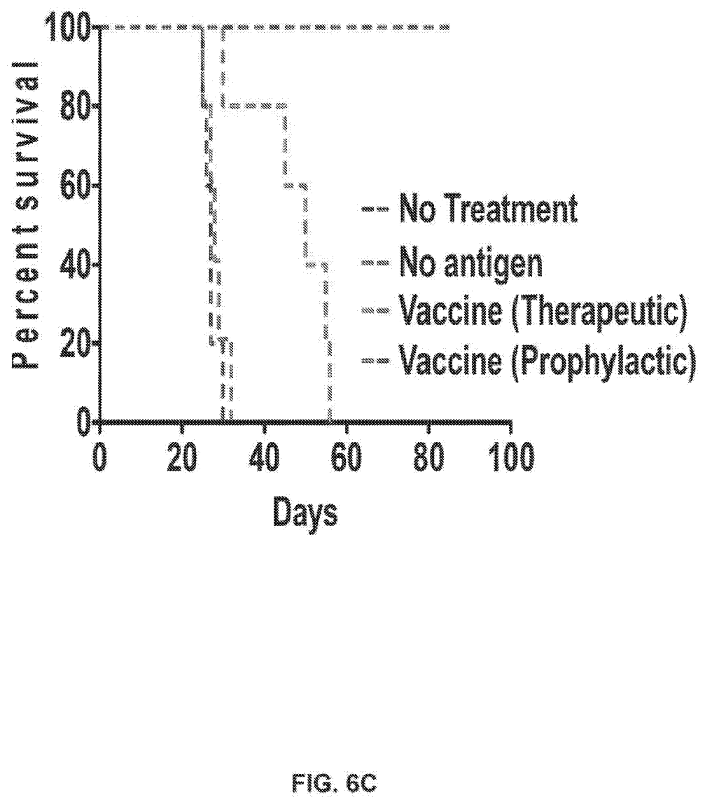

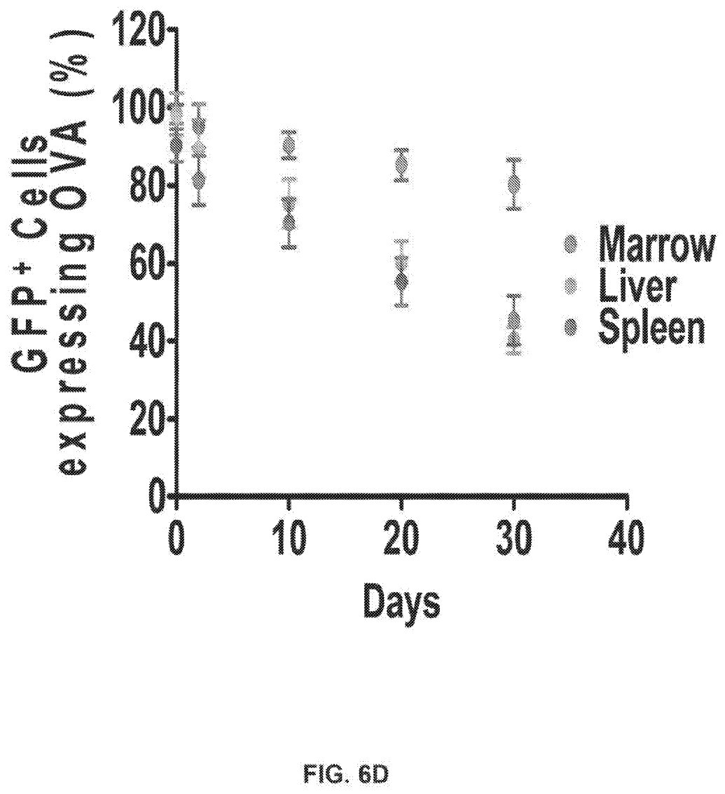

OVA will help optimize an antigen-specific vaccination strategy after a HSCT. The present inventors have established an OVA-expressing acute myeloid leukemia (AML) mouse cell line, containing an MLL-AF9 oncogene, along with the green fluorescent protein (GFP) and luciferase (Luc) reporter genes (FIGS. 6A and 6B). Mice were immunized prophylactically (10 days prior) and therapeutically (7 days after) mounting a challenge with the OVA expressing AML. The subcutaneous vaccine formulation consisted of OVA (100 μg/animal) and a widely used DNA nucleotide based dendritic cell activating factor CpG (100 μg/animal). The present inventors observed full protection after prophylactic vaccination with the prevention of AML cells from engrafting and increased survival after therapeutic immunization (FIG. 6C), and also observed loss of OVA antigen expression in AML cells in therapeutically treated mice, indicating a mechanism of escape from antigen-specific T-cells (FIG. 6D).

The present inventors will challenge the transplanted animals with a bolus dose (˜100 μg) of OVA at pre-determined time intervals after vaccination and monitor CD8+ T-cell response and compare the response across animals that received cryogel vaccine. The antigen-specific CD8+ T-cell population in the different hematopoietic compartments will then be examined. To examine if there is a balanced immune response mediated by both the T- and B-cells (cell-mediated and humoral respectively), the humoral response will be assessed by measuring antibodies titers (IgG1, IgG2) against ovalbumin. These studies will qualitatively assess immune reconstitution and the kinetics of an antigen-specific response.

A second objective of the contemplated studies will be to identify a vaccination protocol to elicit tumor-specific CD8+ T-cell-mediated immune responses that will be sufficiently robust and long-lasting to generate durable tumor regression and/or eradication of AML. In the proposed study, the present inventors will combine the immune reconstitution and vaccination strategies using clinically relevant antigen targets on AML. In particular, AML will be induced in mice using both the OVA/GFP-Luc expressing engineered AML cell line and an untransformed MLL-AF9 cell line. After 2 weeks, a HSCT transplant will be performed after a conditioning regimen following the Zuber protocol (Zuber, et al., Genes & Development 2009, 23 (7): 877-889). Following the transplant, the optimized bone marrow-forming hydrogel scaffold materials described herein will be used to drive lymphocyte reconstitution. Drawing from the results of the vaccination study described above, the present inventors will vaccinate mice post HSCT. In addition to OVA, the use of bone-marrow lysate from AML mice will be explored, as well as clinically relevant leukemia associated antigens (e.g., a peptide of Wilms tumor protein (WT-1) and/or proteinase-3 specific peptide (PR-1)). This could prevent the selection of antigen-loss variants and proliferation of the disease, as observed in the vaccination study described above. The present inventors will also isolate CD8+ T-cells and measure of the levels of secreted cytotoxic granzyme, perforin and interferon-γ after in vitro peptide re-stimulation. Secondary transplants will be performed to determine the leukemic potential of the grafts and to determine if a cure was achieved. Collectively, it is anticipated that the results will indicate that combining immune reconstitution and a clinically relevant antigen-specific immune response would be beneficial in a HSCT.

1. A composition capable of inducing an endogenous immune response to at least one leukemia antigen, comprising
a polymer scaffold comprising open interconnected pores,
a dendritic cell activating factor,
a dendritic cell recruitment factor, and
at least one leukemia antigen.

2. The composition of claim 1, wherein the at least one leukemia antigen is Wilms' Tumor 1 protein (WT-1) or an antigenic fragment thereof.

3. The composition of claim 1, wherein the at least one leukemia antigen is leukemic bone marrow lysate.

4. The composition of claim 1, wherein the at least one leukemia antigen comprises WT-1 H-2Db peptide WT-1₁₂₆₋₁₃₄ (RMFPNAPYL (SEQ ID NO: 1)).

5. The composition of claim 1, wherein the dendritic cell activating factor is CpG.

6. The composition of claim 5, wherein the dendritic cell activating factor is CpG 1826.

7. The composition of claim 1, wherein the dendritic cell recruitment factor is GM-CSF.

8. The composition of claim 1, wherein one or more of the dendritic cell activating factor, dendritic cell recruitment factor, and leukemia antigen are encapsulated by the polymer scaffold.

9. The composition of claim 1, wherein the polymer scaffold comprises polyethylene glycol (PEG) and alginate.

10. The composition of claim 9, wherein the polymer scaffold comprises a molar ratio of PEG to Alginate of about 1:4.

11. The composition of claim 1, wherein the dendritic cell activating factor, the dendritic cell recruitment factor, and the at least one leukemia antigen release from the polymer scaffold over 10 days or less after administration to a subject.

12. The composition of claim 11, wherein a portion of at least one of the dendritic cell activating factor, the dendritic cell recruitment factor, and the at least one leukemia antigen burst release from the polymer scaffold after administration to the subject.

13. A method of manufacturing the composition of claim 1, comprising cryo-polymerization of MA-PEG and MA-Alginate in the presence of one or more of the dendritic cell activating factor, dendritic cell recruitment factor, and leukemia antigen.

14. A method for treating a patient in need thereof, comprising administering the composition of claim 1 to the patient.

15. The method of claim 14, wherein the patient has leukemia.

16. The method of claim 15, wherein the leukemia is Acute Myeloid Leukemia (AML).

17. The method of claim 14, wherein the patient is at risk of developing leukemia.

18. The method of claim 17, wherein the leukemia is AML.

19. The method of claim 14, wherein the patient is in relapse.

20. The method of claim 14, wherein the patient has undergone a procedure selected from a hematopoietic stem cell transplant, a T-cell therapy, and an adaptive immunity regimen.

21. The method of claim 14, further comprising administering one or more anti-cancer agents to the patient.

22. The method of claim 21, wherein the one or more anti-cancer agents are administered prior to administration of the composition.

23. The method of claim 21, wherein the one or more anti-cancer agents are doxorubicin hydrochloride and cytarabine.

24. The method of claim 21, wherein the one or more cancer agents are administered about 1 day before administration of the composition.

25. The method of claim 14, wherein the dendritic cell activating factor, the dendritic cell recruitment factor, and the at least one leukemia antigen release from the polymer scaffold over 10 days or less after administration to the patient.

26. The method of claim 25, wherein a portion of at least one of the dendritic cell activating factor, the dendritic cell recruitment factor, and the at least one leukemia antigen burst release from the polymer scaffold after administration to the patient.

27. The method of claim 14, wherein the composition is administered by subcutaneous injection or implantation.

28. The method of claim 14, wherein administration of the composition induces cytotoxic T lymphocytes specific to leukemia in the patient.

29. The method of claim 14, wherein administration of the composition induces CD11c+ CD86+ activated dendritic cells in the patient.

30. The method of claim 14, wherein administration of the composition induces an adaptive immune response specific to leukemia in the patient.

31. The method of claim 14, wherein administration of the composition reduces or eliminates leukemia cells in the patient.

32. The method of claim 14, wherein administration of the composition prevents or reduces the likelihood of a future occurrence of leukemia.

33. The method of claim 14, wherein administration of the composition does not cause pancytopenia or autoimmunity in the subject.

34. The method of claim 14, wherein the composition is administered one time to the patient.

35. A method for preventing and/or reducing the incidence of leukemia in a subject, comprising transplanting bone marrow or hematopoietic stem cells from a donor to the subject, wherein the donor has been administered the composition of claim 1.

36. The method of claim 35, wherein the subject has undergone myeloablation therapy prior to transplant.

37. A kit comprising the composition of claim 1.
